SEQUENCE LISTING (6 sequences)

SEQ ID NO: 1 - DNA, 15 bp, Artificial Sequence
  G12D
tacgccatcagctcc

SEQ ID NO: 2 - DNA, 15 bp, Artificial Sequence
  G12R
tacgccacgagctcc

SEQ ID NO: 3 - DNA, 15 bp, Artificial Sequence
  G12V
tacgccaacagctcc

SEQ ID NO: 4 - DNA, 41 bp, Artificial Sequence
  G12D with mutations centered (IDT DNA)
acttgtggtagttggagctgatggcgtaggcaagagtgcct

SEQ ID NO: 5 - DNA, 41 bp, Artificial Sequence
  G12R with mutations centered (IDT DNA)
acttgtggtagttggagctcgtggcgtaggcaagagtgcct

SEQ ID NO: 6 - DNA, 41 bp, Artificial Sequence
  G12V with mutations centered (IDT DNA)
acttgtggtagttggagctgttggcgtaggcaagagtgcct

(19) United States
(12) Patent Application Publication
(10) Pub. No.: US 2023/0279506 A1
(43) Pub. Date: Sep. 7, 2023

(54) SYSTEMS AND METHODS FOR CELL CAPTURE, BIOMARKER DETECTION, AND CONTACT-FREE CELL LYSIS

(71) Applicant: Trustees of Dartmouth College, Hanover, NH (US)

(72) Inventors: Amogha Tadimety, Hanover, NH (US); Alison Burklund, Hanover, NH (US); Timothy J. Palinski, Hanover, NH (US); John X.J. Zhang, Hanover, NH (US)

(73) Assignee: Trustees of Dartmouth College, Hanover, NH (US)

(21) Appl. No.: 18/015,627

(22) PCT Filed: Jul. 13, 2021
(86) PCT No.: PCT/US2021/041431

§ 371 Date: Jan. 11, 2023

Related U.S. Application Data
(60) Provisional application No. 63/051,145, filed on Jul. 13, 2020.

(51) Int. Cl. C12Q 1/689; B01L 3/00; C12Q 1/6834

(52) U.S. Cl. CPC C12Q 1/689; B01L 3/502761; C12Q 1/6834; B01L 2200/0668; C12Q 1/6804

(57) ABSTRACT

In an embodiment, the present disclosure pertains to a method of detecting an analyte from vesicles in a sample. In an additional embodiment, the present disclosure pertains to an analyte detection platform. In a further embodiment, the present disclosure pertains to a sensor. In another embodiment, the present disclosure pertains to a method of detecting an analyte from a sample. In an additional embodiment, the present disclosure pertains to a method of lysing vesicles. In a further embodiment, the present disclosure pertains to a vesicle lysis platform.

### CROSS-REFERENCE TO RELATED APPLICATIONS

This application claims priority to U.S. Provisional Pat. Application No. 63/051,145, filed on Jul. 13, 2020. The entirety of the aforementioned application is incorporated herein by reference.

### BACKGROUND

Currently available methods relating to analyte detection from vesicles in samples, analyte detection platforms, sensors, and analyte detection from a sample suffer from numerous drawbacks, that can include, without limitation, slow processing time, limited analyte sensitivity, and complicated equipment. Additionally, currently available systems and methods for lysing vesicles and vesicle lysis platforms suffer similar disadvantages. Various embodiments of the present disclosure address the aforementioned limitations.

### SUMMARY

In an embodiment, the present disclosure pertains to a method of detecting an analyte from vesicles in a sample. Such methods generally include one or more of the following steps of: (a) flowing the sample through a platform, where vesicle capture particles bind to the vesicles in the sample to form particle-vesicle complexes and the particle-vesicle complexes become immobilized on a first surface of the platform; (b) lysing the vesicles of the particle-vesicle complexes, thereby releasing the analyte; (c) associating the analyte with an analyte detecting agent, where the analyte detecting agent is immobilized on a second surface of the platform; and (d) detecting the analyte. In some embodiments, the detecting can include detecting a change in property of the second surface and correlating the change in property of the second surface to a characteristic of the analyte.

In a further embodiment, the present disclosure pertains to a platform for analyte detection in a sample. In some embodiments, the platform can include an inlet region for receiving a sample, a mixing region for mixing the sample, a capturing region including a first surface for capturing one or more components of the sample, where the first surface is downstream the mixing region, and a sensing region including a second surface for detecting an analyte from the sample. In some embodiments, the second surface includes an analyte detecting agent.

In an additional embodiment, the present disclosure pertains to sensors used for analyte detection. In some embodiments, the sensor includes a surface for detecting an analyte from a sample. In some embodiments, the surface includes a dielectric surface and nanostructures randomly oriented on the dielectric surface. In some embodiments, the nanostructures are coupled to an analyte detecting agent.

In another embodiment, the present disclosure pertains to a method of detecting an analyte from a sample. Such methods generally include one or more of the following steps of: (a) flowing the sample through a sensor; and (b) detecting the analyte. In some embodiments, the sensor includes a surface for detecting an analyte from a sample. In some embodiments, the surface includes a dielectric surface and nanostructures randomly oriented on the dielectric surface. In some embodiments, the nanostructures are coupled to an analyte detecting agent. In some embodiments, the detecting includes detecting a change in property of the surface, and correlating the change in property of the surface to a characteristic of the analyte.

In further embodiments, the present disclosure relates to methods of contract-free vesicle lysis. Such methods generally include one or more of the following steps of: (a) flowing the sample through a platform, where vesicle capture particles bind to the vesicles in the sample to form particle-vesicle complexes and the particle-vesicle complexes become immobilized on a surface of the platform; and (b) lysing the vesicles of the particle-vesicle complexes. In some embodiments, the surface includes a magnetic surface. In some embodiments, the lysing includes exposing the surface to an alternating magnetic field (AMF). In some embodiments, the AMF heats the magnetic surface and thereby generates heat. In some embodiments, the generated heat lyses the vesicles of the particle-vesicle complexes.

In an additional embodiment, the present disclosure pertains to contact-free vesicle lysis systems. In some embodiments, a vesicle lysis platform includes a surface. In some embodiments, the surface includes a magnetic surface.

### DESCRIPTION OF THE DRAWINGS

FIGS. 1A1 and 1A2 illustrate an analyte detection platform according to an aspect of the present disclosure.

FIG. 1B illustrates a method of detecting an analyte from vesicles in a sample according to an aspect of the present disclosure.

FIG. 1C illustrates a sensor according to an aspect of the present disclosure

FIG. 1D illustrates a method of detecting an analyte from a sample according to an aspect of the present disclosure.

FIG. 1E illustrates a method of lysing vesicles according to an aspect of the present disclosure.

FIG. 1F illustrates a vesicle lysis platform according to an aspect of the present disclosure.

FIG. 2 illustrates a schematic of immunomagnetic capture and plasmonic detection system. Cross-sectional schematic of immunomagnetic bacterial enrichment working principle (left) and working principle of nano-scale plasmonic sensing platform (right).

FIGS. 3A-3B illustrate capture efficiency and plasmonic sensing results. FIG. 3A shows capture efficiency of S. aureus in whole blood matrix. PC = percent capture (mean). FIG. 3B shows plasmonic sensing results of S. aureus cell lysate. Peak absorbance wavelength shifts as a function of nucleic acid concentration.

FIGS. 4A-4C illustrate a schematic of an integrated capture and detection microsystem: (1) bacterial capture from whole blood; (2) cell lysis; and (3) DNA detection on a single micro-chip. The microsystem in this illustration represents an integrated single-chip platform according to aspects of the present disclosure.

FIG. 5A1-5C illustrate an overview of an integrated microsystem. FIG. 5A1-5A3 show chip functionality. Bacterial samples (FIG. 5A1) and functionalized magnetic nanoparticles (MNPs) (FIG. 5A2) are pushed through micro-chip in parallel. Mixing and incubation occur throughout the jagged serpentine microchannel (FIG. 5A3). Bacteria-MNP complexes (FIG. 5B4) are isolated in the hexagonal microchamber using an external magnet (FIG. 5B5). Bacteria are thermally lysed (FIG. 5B6). The novel localized surface plasmon resonance (LSPR) sensor (FIG. 5B7) is exposed to bacterial lysate. Upon nucleic acid hybridization to sensor, a red shift in the peak absorbance is observed (FIG. 5B8). FIG. 5C shows sample processing workflow and timeline. 12 min is required for bacterial enrichment (100 µL/min, 1 mL sample), 10 min is required for bacterial lysis, and 5 min is required for nucleic acid sensing. A total of 3 min is required for fluid manipulation (i.e., air, phosphate-buffered saline (PBS)). Total-analytical-time for the integrated enrichment and detection platform is 30 min.

FIGS. 6A-6B illustrate a microfluidic immunomagnetic bacterial capture. FIG. 6A shows bacterial capture efficiency as a function of bacterial species (S. aureus, P. aeruginosa) and input bacterial concentration. X-axis is presented as a logarithmic scale. Standard error of the mean is reported, n = 3 samples per condition. FIG. 6B shows capture antibody specificity. Input bacterial concentration is approximately 10⁵ CFU/mL for all reported data series. Bacteria samples processed without MNPs (dark gray) represent the average observed bacterial loss within the microsystem of three independently evaluated bacterial species: S. aureus, P. aeruginosa, and E. coli (n = 3 per bacterial species, n = 9 total). Standard error of the mean is reported.

FIGS. 7A-7F illustrate a nanoplasmonic sensing of bacterial nucleic acids. FIG. 7A shows representative extinction spectra. Following conjugation of peptide nucleic acid (PNA) probes to gold nanoparticles a red shift is observed. An additional red shift is observed upon hybridization of target nucleic acids to PNA probes are observed. The magnitude of this second peak wavelength shift represents the signal of interest. FIGS. 7B-7D shows peak wavelength shift as a function of input bacterial load for (FIG. 7B) S. aureus, (FIG. 7C) P. aeruginosa, and (FIG. 7D) E. coli. FIGS. 7E-7F show probe specificity characterizations of a P. aeruginosa probe exposed to (FIG. 7E) E. coli cell lysate, and (FIG. 7F) S. aureus cell lysate. Standard error of the mean is reported.

FIGS. 8A-8C illustrate data reproducibility on nanoplasmonic sensors for (FIG. 8A) S. aureus, (FIG. 8B) E. coli, and (FIG. 8C) P. aeruginosa. For all nanoplasmonic sensing studies, data was collected using 3 biological samples on 3 different sensor devices. Each device was exposed to a unique bacterial lysate sample, and three measurements were taken with each device. The mean and standard error of the mean are reported. The data in FIGS. 7B-7D represent all 9 measurements combined.

FIGS. 9A-9B illustrate performance of integrated bacterial enrichment and detection platform. FIG. 9A shows magnitude of peak wavelength shift with integrated enrichment and without enrichment as a function of input bacterial concentration; n = 3 samples per condition. FIG. 9B shows observed signal enhancement factor using integrated microsystem as a function of input bacterial concentration. Standard error of the mean is reported.

FIG. 10 illustrates data reproducibility for integrated bacterial enrichment and nanoplasmonic detection. Each listed sample represents a unique biological sample processed on the system. Each unique biological sample was evaluated on three different sensors. The mean and standard error of the mean of the sensing output for each unique sample are reported. The data in FIG. 9A represent all 9 measurements combined.

FIGS. 11A-11B illustrate a multiplexed capture and detection of polymicrobial samples. FIG. 11A shows a table reporting peak shift as a function of input sample composition. FIG. 11B show magnitude of peak wavelength shift in single species samples (i.e., S. aureus) versus polymicrobial samples (i.e., S. aureus + P. aeruginosa) as a function of bacterial concentration. Standard error of the mean is reported; n = 3 samples per condition.

FIG. 12 illustrates workflow for device fabrication (1-2) and operation (3-4). Shown are (1) Bidirectional microfluidic printing for dispersion of bare gold nanorods into sensing spots; (2) Sequence-specific conjugation with PNA probes for 3 clinically relevant mutations; (3) Attachment of microfluidic device to deliver sample, circulating tumor DNA (ctDNA) will bind to PNA probes if present; and (4) Measure of absorbance spectrum through each spot to measure bound ctDNA concentration to probes.

FIGS. 13A-13E illustrate images of fabricated nanorod spots and associated spectra. FIG. 13A shows optical image of fabricated nanorod spots. FIG. 13B shows scanning electron microscope (SEM) image of fabricated nanorod spots. FIG. 13C shows zoom-in SEM showing dispersion of nanorods. FIG. 13D shows multiple nanorod spots on single chip for multiplexing. FIG. 13E shows parameters for nanorod printing.

FIGS. 14A-14B illustrate conjugation workflow and associated spectra. FIG. 14A shows workflow for conjugation starting from bare gold nanorods dispersed on glass slide. First step is activation of the gold followed by a wash and coupling with the PNA probe. FIG. 14B shows associated extinction spectra of the bare rods and the rods after conjugation, showing an approximate 20 nm shift in the peak wavelength after successful coupling (779 nm when bare, 808 nm after conjugation).

FIG. 15A-15C3 illustrate two dimensional (2D) Electromagnetic Conformal Layer Simulation. FIG. 15A shows simulated extinction spectra of bare gold nanorod, PNA-conjugated gold nanorods, and PNA-DNA bound gold nanorods. FIG. 15B shows spectral zoom-in of peak resonance features, demonstrating a large peak shift after PNA conjugation to the nanorods and then a smaller shift upon DNA binding. FIG. 15C1-C3 show images of simulation setup including bare rod, conformal layers, and simulation plane.

FIGS. 16A-16C illustrate sensing curves for 3 different point mutations in KRAS gene, the G12D, G12R, and G12V variants. Peak shift is calculated as the difference between peak wavelengths at each concentration and without ctDNA. Each data point represents measurements on three devices conjugated and put in contact with that sequence. Error bars represent standard error of the mean. FIG. 16A shows sensing of G12D synthetic oligos. FIG. 16B shows sensing of G12R synthetic oligos. FIG. 16C shows sensing of G12V synthetic oligos.

FIGS. 17A-17D illustrates multiplexed sensing of 3 mutations in the KRAS gene. Peak wavelength shift is calculated as the difference between peak wavelength before and after ctDNA addition. Each data point represents measurements on three sensing spots conjugated and put in contact with relevant targets. Error bars represent standard error of the mean. FIG. 17A shows sensing measurement of all three conjugated spots, with only G12V synthetic DNA present. FIG. 17B shows mixed sample of G12V and G12D variant showing no binding to G12R sensor. FIG. 17C shows mixed samples of all three variants showing approximately equal binding. FIG. 17D shows mixed samples of G12D and G12R synthetic DNA showing semi-quantitative discrimination between wavelength output.

FIG. 18A1-18C2 illustrate an overview of a proposed detection mechanism. FIG. 18A1-A5 show a microchip design showing Phase I focus on the capture and transduction of RNA binding. FIG. 18B shows that initially nanoparticles are tethered to the gold film by PNA probes. If severe acute respiratory syndrome coronavirus 2 (SARS-CoV-2) RNA is present, binding will occur, and shorten the length of the tether. FIG. 18C1-C2 show that if PNAs are unbound, the longer tether remains out of the plasmonic electric field decay length, but if PNAs bind to target RNA, the tether shortens, plasmonic coupling occurs, and binding can be visualized on dark field image.

FIG. 19A1-19B illustrate a nanoparticle-on-film simulation overview. FIG. 19A1-19A3 show three geometries of nanoparticles to be tested: nanocube, nanosphere, and nanorod. FIG. 19B shows preliminary CST simulation data, showing extremely high quality resonances with large peak shifts (hundreds of nm) from small (2-10 nm) thickness changes.

FIG. 20A1-20B illustrate an overview schematic of bacterial enrichment and contact-free lysis driven by an AC magnetic field. FIG. 20A1-20A2 (Step 1): The syringe pump pushes sample through hexagonal micro- channel. The external magnet retains bacteria bound to functionalized magnetic nanoparticles within the microchannel, while waste products are collected as the output. TEM image of S. aureus (~0.5 µm) bound to magnetic nanoparticles (~150 nm) is shown in FIG. 20A2. FIG. 20B (Step 2): Overview schematic of contact-free cell lysis. External magnet is removed, microchip is placed in coil, and microchip is exposed to an AMF. Bacteria are thermally lysed, enabling downstream nucleic acid collection and analysis.

FIG. 21A1-21C2 illustrate an overview of a device substrate and heating mechanism. FIG. 21A1-21A3 show a magnetic polymer microchip. Substrate modification consists of three identical spin coated polymer layers (P-1-P-3). Magnetic nanoparticles mixed within the polymer (PDMS) enable thermal lysis of bacteria, making molecules of interest available (i.e., DNA) for analysis (FIG. 21A2). Shown in FIG. 21A3 is an atomic force microscopy image (AFM) displaying topography of a magnetic polymer surface. FIG. 21B shows an image of magnetic polymer-coated microchip in microfluidic cartridge. FIG. 21C1-C2 shows a schematic of heating mechanism for magnetic nanoparticles embedded in a polymer matrix (FIG. 21C1). Néel relaxation-the rapid change in magnetic moment in opposition to the nanoparticle’s crystal-line structure-drives heat generation (FIG. 21C2).

FIG. 22A1-22D illustrate microfluidic immunomagnetic bacterial capture. FIG. 22A1-A2 show Transmission Electron Microscopy (TEM) images of S. aureus bound to 150 nm magnetic nanoparticles. FIG. 22B shows bacterial capture efficiency as a function of flow rate. FIG. 22C shows bacterial capture efficiency as a function of magnetic nanoparticle mass. FIG. 22D shows bacterial capture efficiency as a function of cell concentration. Control samples contained no functionalized magnetic particles and were evaluated to account for any potential bacterial loss and/or gain within the micro-system. All samples were evaluated in triplicate. Standard error of mean is reported.

FIGS. 23A-23B illustrate magnetic polymer microchip heating. FIG. 23A shows representative thermal image of microchip in coil after 30 s exposure to AMF. FIG. 23B shows temperature of the microchip as a function of time. Temperature data were collected using a thermal camera. Three unique devices were evaluated, and each device was tested in triplicate. Standard error of the mean is reported.

FIGS. 24A-24B illustrate recovered DNA and cell viability. FIG. 24A shows total recovered DNA and FIG. 24B shows cell death as a function of cell load following 60 s exposure to AMF. All samples were evaluated in triplicate, with three unique devices used. Standard error of mean is reported.

FIGS. 25A-25B illustrate bacterial capture efficiency optimization. FIG. 25A shows bacterial capture efficiency as a function of flow rate. Using Applicants’ microfluidic chip, relatively high flow rates could be achieved, while preserving capture efficiency. Flowrate experiments were conducted at bacterial load on the order of 10³ CFU/mL, and with 25 µg functionalized magnetic nanoparticles. Experiments were performed in triplicate, and standard error of the mean is reported. FIG. 25B shows bacterial capture efficiency as a function of magnetic nanoparticle (MNP) mass. Increased MNP mass resulted in significantly greater bacterial capture efficiency. MNP mass optimization experiments were conducted at bacterial load on the order of 10³ CFU/mL, and at a flowrate of 10 mL/h. Experiments were performed in triplicate, and standard error of the mean is reported.

FIGS. 26A-26B illustrate magnetic polymer characterization and optimization. FIG. 26A shows characterization of specific absorbance rate of the iron oxide heating particles as a function of field frequency. SAR was characterized in water. FIG. 26B shows examples of various multilayer magnetic polymer substrates (left to right: 1-layer, 2-layer, 3-layer, 5- layer).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Current methods relating to analyte detection from vesicles (e.g., cells) in samples, analyte detection platforms, sensors, and analyte detection from a sample contain numerous drawbacks such as, but not limited to, slow processing time, limited sensitivity, and complicated equipment. In addition, currently available systems and methods for lysing vesicles have similar drawbacks.

Accordingly, a need exists for more effective systems and methods for analyte detection from vesicles in samples, analyte detection platforms, sensors, and analyte detection from a sample. Furthermore, a need exists for more effective systems and methods for lysing vesicles. Various embodiments of the present disclosure address the aforementioned limitations.

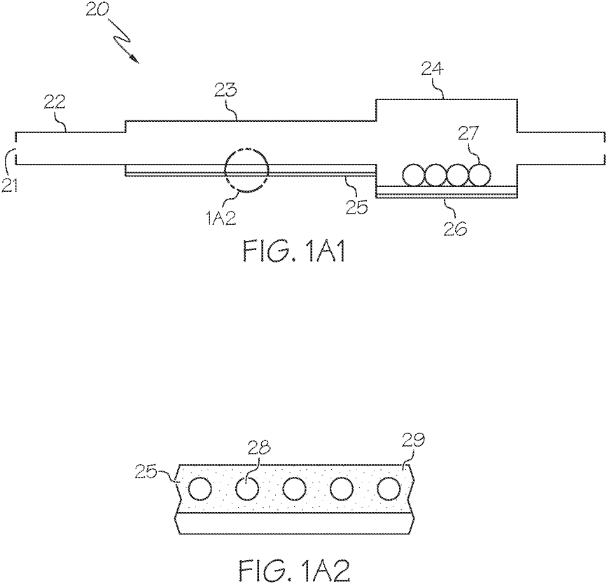

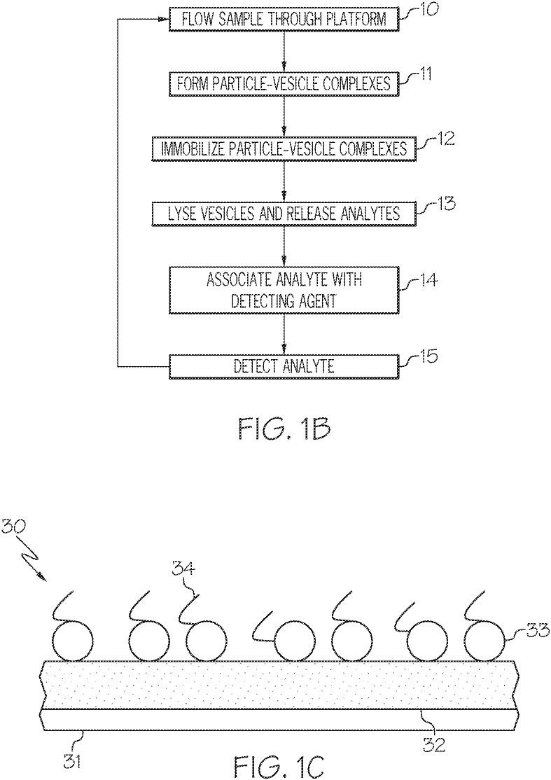

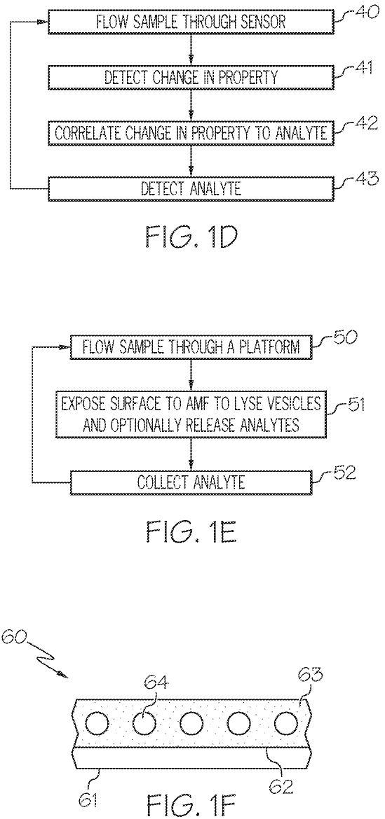

In some embodiments, the present disclosure pertains to an analyte detection platform. In some embodiments illustrated in FIG. 1A1, the analyte detection platform is in the form of platform 20, which includes an inlet region 21 for receiving a sample, a mixing region 22, a capture region 23, and a sensing region 24. As illustrated in FIG. 1A1, the capture region 23 has a first surface 25 for capturing one or more components of the sample, where the first surface 25 is downstream the mixing region 22. As also shown in FIG. 1A1, the sensing region 24 includes a second surface 26 for detecting an analyte from the sample, where the second surface 26 includes analyte detecting agents 27.

In a non-limiting embodiment, illustrated in FIG. 1A2, the first surface 25 is a magnetic surface. In some embodiments, the magnetic surface includes magnetic particles 28 associated with polymers 29.

In some embodiments, the analyte detection platforms of the present disclosure may be utilized to detect analytes from vesicles in a sample in accordance with the analyte detection methods of the present disclosure. For instance, in some embodiments, a sample containing vesicles and vesicle capture particles may flow through inlet region 21 of platform 20 and into mixing region 22, where vesicle capture particles bind to vesicles and form particle-vesicle complexes. Thereafter, the particle-vesicle complexes flow into capture region 23, where they become immobilized on first surface 25 through various mechanisms as described herein.

Next, the immobilized vesicles in the sample are lysed on first surface 25, thereby releasing the analyte from the vesicles. Vesicle lysis may also occur through various mechanisms as described herein. For instance, in some embodiments, an alternating magnetic field (AMF) may be applied to the first surface 25 (e.g., to a magnetic surface shown in FIG. 1A2), thereby heating first surface 25 and causing lysis of the immobilized vesicles without any contact between the vesicles and first surface 25. In some embodiments, the surface is capable of generating heat upon exposure to AMF.

The released analytes then flow through sensing region 24, where they become associated with analyte detecting agents 27 on second surface 26. The analytes are then detected through detecting a change in property of second surface 26 and correlating the change in the property to a characteristic of the analyte.

In some embodiments, the present disclosure pertains to methods of detecting an analyte from vesicles in a sample. In some embodiments illustrated in FIG. 1B, the methods of the present disclosure include one or more of the following steps of flowing the sample through a platform (step 10), forming particle-vesicle complexes when vesicle capture particles bind to the vesicles in the sample (step 11), immobilizing the particle-vesicle complexes (step 12), lysing the vesicles of the particle-vesicle complexes and thereby releasing the analyte (step 13), associating the analyte with an analyte detecting agent (step 14), and detecting the analyte (step 15).

In some embodiments, the analyte detection platforms of the present disclosure (e.g., analyte detection platform 20 shown in FIG. 1A1) can be utilized to practice the analyte detection methods of the present disclosure. In some embodiments illustrated herein, the analyte detection steps of the present disclosure can have additional embodiments.

For instance, in some embodiments, step 10 (i.e., flowing the sample through a platform) includes introducing a sample into an inlet region of a platform. The sample may include vesicles containing analytes. In some embodiments, the sample may contain vesicles and vesicle capture particles. In some embodiments, the vesicles and the vesicle capture particles may be separately introduced to the inlet region. In some embodiments, the vesicles and the vesicle capture particles may be pre-mixed to form the sample prior to introducing into the platform. In some embodiments, the vesicles and the vesicle capture particles may be introduced via separate inlets of a platform and mixed downstream in the platform.

In some embodiments, step 11 (i.e., forming particle-vesicle complexes) involves vesicle capture particles binding to the vesicles. In some embodiments, the particle-vesicle complexes may be formed prior to introducing the sample into the platform (such as when the vesicles and the vesicle capture particles are pre-mixed to form the sample). In some embodiments, the particle-vesicle complexes may be formed following introducing the vesicles and the vesicle capture particles into the platform.

In some embodiments, step 12 (i.e., immobilizing the particle-vesicle complexes) involves immobilization of the particle-vesicle complexes on a first surface of the platform. In some embodiments, immobilization may be achieved by a magnetic force between the first surface and the complexes. In some embodiments, immobilization may be achieved through biomolecular binding or electrostatic interaction.

In some embodiments, step 13 (i.e., lysing the vesicles of the particle-vesicle complexes, thereby releasing the analytes) involves breaking open the vesicles to release analytes. In some embodiments, this may be achieved through exposing a surface that is in the form of a microchip to an alternating magnetic field. In some embodiments, this may be achieved through heating the vesicles or putting them in contact with a chemical detergent or biological enzyme.

In some embodiments, step 14 (i.e., associating released analytes with analyte detecting agents) occurs when an analyte detecting agent is immobilized on a second surface of the platform. In some embodiments, the analyte associates with the analyte detection agent through biomolecular interaction, complementary hybridization, or electrostatic interaction.

In some embodiments, step 14 (i.e., detecting the analyte) includes, for example, detecting a change in property of the second surface and correlating the change in property of the second surface to a characteristic of the analyte. In some embodiments, the method can be continuous and/or repeated until all analytes have been detected.

Additional embodiments of the present disclosure pertain to sensors. In some embodiments illustrated in FIG. 1C, the sensors of the present disclosure may be in the form of sensor 30, which includes a surface 31 for detecting an analyte from a sample. As illustrated in FIG. 1C, the surface 31 includes a dielectric surface 32 and nanostructures 33 randomly oriented on the dielectric surface 32. As further illustrated in FIG. 1C, the nanostructures 33 are coupled to analyte detecting agents 34. In some embodiments, the sensor is a plasmonic sensor.

Further embodiments of the present disclosure pertain to methods of detecting an analyte from a sample through sensing, such as through the utilization of sensors 30 illustrated in FIG. 1C. In some embodiments, the sensing is plasmonic sensing. In some embodiments illustrated in FIG. 1D, the methods of the present disclosure include a step of flowing the sample through a sensor (step 40) (e.g., sensor 30). In some embodiments, the sensor includes a surface (e.g. surface 31) for detecting an analyte from a sample. In some embodiments, the surface includes a dielectric surface (e.g., dielectric surface 32) and nanostructures (e.g., nanostructures 33) randomly oriented on the dielectric surface. In some embodiments, the nanostructures are coupled to an analyte detecting agent (e.g., analyte detecting agents 34).

As illustrated in FIG. 1D, the methods of the present disclosure can further include the steps of detecting a change in property of the surface of the sensor (step 41), correlating the change in property of the surface to a characteristic of the analyte (step 42), and detecting the analyte (step 43). In some embodiments, the method can be continuous and/or repeated until all analytes have been detected.

Further embodiments of the present disclosure pertain to methods of contact-free vesicle lysis. In some embodiments illustrated in FIG. 1E, the method of lysing vesicles in a sample generally involves one or more of the following steps of flowing the sample through a platform (step 50), and exposing a surface of the platform to an alternating magnetic field (AMF) to lyse the vesicles (step 51). In some embodiments, the contact-free vesicle lysis methods of the present disclosure result in the release of analytes from the vesicles (step 51), and the subsequent collection of the analytes (step 52).

In some embodiments, vesicle capture particles bind to the vesicles in the sample to form particle-vesicle complexes. In some embodiments, the particle-vesicle complexes become immobilized on the surface of the platform. In some embodiments, the surface is a magnetic surface. In some embodiments, the magnetic surface includes a polymer and magnetic particles associated with the polymer. In some embodiments, the AMF heats the surface (e.g., a magnetic surface) and thereby generates heat, and the generated heat lyses the vesicles of the particle-vesicle complexes. In some embodiments, the surface is capable of generating heat upon exposure to AMF. In some embodiments, the surface is capable of generating heat upon exposure to AMF. In some embodiments, the method can be continuous and/or repeated until all vesicles have been lysed.

Additional embodiments of the present disclosure pertain to contact-free vesicle lysis systems. In some embodiments illustrated in FIG. 1F, the contact-free vesicle lysis systems of the present disclosure include a vesicle lysis platform 60, which includes a surface 61. In some embodiments, the surface 61 includes magnetic surface 62. In a non-limiting embodiment, magnetic surface 62 can include polymers 63 and magnetic particles 64 associated with polymers 63.

In some embodiments, the contact-free vesicle lysis systems of the present disclosure may be utilized to lyse cells in accordance with the contact free cell lysis methods of the present disclosure. For instance, in a specific embodiment, a sample containing vesicles and vesicle capture particles may flow through vesicle lysis platform 60, where the formed particle-vesicle complexes become immobilized on the surface 61 through various mechanisms, such as magnetic immobilization, biomolecular binding, or electrostatic interaction.

For example, in some embodiments, the surface 61 includes a magnetic surface 62. In this example, the formed particle-vesicle complexes become immobilized on magnetic surface 62. Thereafter, the magnetic surface 62 is exposed to AMF, which heats the magnetic surface 62 and thereby generates heat. Thereafter, the generated heat lyses the vesicles of the particle-vesicle complexes.

The contact-free vesicle lysis systems may be used to release the analyte from the vesicle for further analysis by other systems as well. As illustrated above, in some embodiments, the surface is capable of generating heat upon exposure to AMF.

As set forth in more detail herein, the systems and methods of the present disclosure can have numerous embodiments. For instance, the methods for detecting analytes from vesicles in a sample can utilize various sample processing steps, samples, flowing methods, vesicles, vesicle capture particles, immobilization methods, lysing methods, and analyte detecting agents. Moreover, the methods of the present disclosure can utilize various changes in properties to detect numerous types of analytes.

Furthermore, various platforms may be utilized to lyse vesicles and detect analytes from the lysed vesicles. For instance, the platforms can include various inlet regions, capturing regions, and sensing regions in various arrangements. In addition, the platforms of the present disclosure can utilize various analyte detecting agents, surfaces, and platform configurations.

Additionally, various sensors and sensing methods may be utilized to detect various analytes from various samples. For example, the sensors of the present disclosure can include various dielectric surfaces and nanostructures in various orientations. In addition, the sensors of the present disclosure can utilize numerous analyte detecting agents and have various configurations.

Additionally, the present disclosure may utilize various contact-free vesicle lysis platforms and contact-free vesicle lysis methods. For instance, as set forth in further detail herein, the contact-free vesicle lysis platforms and methods of the present disclosure can utilize various surfaces, for example magnetic surfaces, that can include, without limitation, numerous polymers and magnetic particles. In addition, the methods and platforms of the present disclosure can lyse numerous types of vesicles from various samples. The methods and platforms of the present disclosure can also utilize various flowing methods, vesicle capture particles, and surfaces

### Analyte Detection From Vesicles in a Sample

As disclosed in further detail herein, embodiments of the present disclosure pertain to methods of detecting an analyte from vesicles in a sample. Such methods generally include one or more of the following steps of: (a) flowing the sample through a platform, where vesicle capture particles bind to the vesicles in the sample to form particle-vesicle complexes, and where the particle-vesicle complexes become immobilized on a first surface of the platform; (b) lysing the vesicles of the particle-vesicle complexes, thereby releasing the analyte; (c) associating the analyte with an analyte detecting agent, where the analyte detecting agent is immobilized on a second surface of the platform; and (d) detecting the analyte. In some embodiments, the detecting can include detecting a change in property of the second surface and correlating the change in property of the second surface to a characteristic of the analyte.

### Additional Sample Processing Steps

As set forth in further detail herein, the methods of the present disclosure can include additional sample processing steps. For example, in some embodiments, the method further includes clearing the sample from the platform after step (a). In some embodiments, the method further includes clearing excess or unwanted portions of the sample from the platform. In some embodiments, the method further includes the step of removing excess fluid from the platform.

In some embodiments, the method further includes the step of introducing a carrier liquid to the first surface of the platform before the lysing in step (b). In some embodiments, the carrier liquid can include, without limitation, phosphate-buffered saline (PBS), TE buffer, alcohols, water-based solutions, and combinations thereof. In some embodiments, the analyte is released into the carrier liquid to form a lysate during the lysing in step (b).

In some embodiments, the method further includes the step of flowing and exposing the lysate to the second surface of the platform after step (b). In some embodiments, step (b) further includes incubating the lysate with the second surface and then clearing the lysate from the platform.

### Samples

As set forth in further detail herein, the methods of the present disclosure can detect analytes from vesicles in numerous types of samples. For example, in some embodiments, the sample can include, without limitation, a biological sample obtained from a subject, an environmental sample obtained from an environment, and combinations thereof.

In some embodiments, the sample includes a biological sample obtained from a subject. In some embodiments, the biological sample can include, without limitation, a blood sample, a tissue sample, a urine sample, a saliva sample, a sputum sample, a swab sample, a swab sample put into a carrier solution, a processed blood sample, and combinations thereof.

In some embodiments, the sample includes an environmental sample. In some embodiments, the environmental sample can include, without limitation, a food sample, a water sample, a swab sample, a swab sample put into a carrier solution, a surface swab sample, a passive material sample put into a carrier solution, and combinations thereof.

### Flowing the Samples

As set forth in further detail herein, the methods of the present disclosure can utilize numerous methods for flowing the sample through the platform. For instance, in some embodiments, the flowing includes flowing the sample through the platform along with the vesicle capture particles. In some embodiments, the sample is co-introduced into the platform along with the vesicle capture particles. In some embodiments, the sample is pre-incubated with the vesicle capture particles prior to co-introduction into the platform.

In some embodiments, the flowing can include flowing the sample through the platform while the vesicle capture particles are immobilized on the first surface of the platform. In some embodiments, the vesicle capture particles are pre-immobilized on or part of the first surface.

In some embodiments, the methods of the present disclosure can further include a step of immobilizing the vesicle capture particles on the first surface prior to the flowing step. In some embodiments, the flowing occurs through a method that can include, without limitation, pumping, mechanical pumping, electrical pumping, syringe-facilitated flow, pipette-facilitated flow, capillary flow, peristaltic flow, pressure-driven flow, and combinations thereof.

### Vesicles

As outlined in further detail herein, the methods of the present disclosure can detect analytes from various vesicles. For instance, in some embodiments, the vesicles can include, without limitation, viruses, bacteria, yeast, fungi, prokaryotic cells, eukaryotic cells, extracellular vesicles, and combinations thereof. In some embodiments, the vesicles include viruses. In some embodiments, the vesicles include severe acute respiratory syndrome coronavirus 2 (SARS-CoV-2). In some embodiments, the vesicles include Human Papilloma Virus (HPV).

In some embodiments, the vesicles include eukaryotic cells. In some embodiments, the eukaryotic cells include cancer cells. In some embodiments, the vesicles include bacteria.

In some embodiments, the vesicles include extracellular vesicles. In some embodiments, the extracellular vesicles include exosomes.

### Analytes

As set forth in further detail herein, various analytes can be detected via the methods of the present disclosure. For example, in some embodiments, the analyte can include, without limitation, nucleotides, oligonucleotides, RNA, DNA, ribosomal RNA (rRNA), messenger RNA (mRNA), microDNA, microRNA, extrachromosomal circular DNA (eccDNA), circulating tumor DNA (ctDNA), small molecules, proteins, mutated versions thereof, and combinations thereof.

In some embodiments, the analyte includes RNA. In some embodiments, the analyte includes mutated nucleotides. In some embodiments, the analyte includes wild-type nucleotides.

### Vesicle Capture Particles

As detailed herein, the methods of the present disclosure can utilize various vesicle capture particles in numerous manners. For instance, in some embodiments, the vesicle capture particles are immobilized on the first surface of the platform prior to the flowing step. In some embodiments, the vesicle capture particles are lyophilized on the first surface of the platform prior to the flowing step.

In some embodiments, the vesicle capture particles can include, without limitation, metal particles, magnetic particles, polymer-based particles, gelled particles, and combinations thereof. In some embodiments, the vesicle capture particles include magnetic particles.

In some embodiments, the vesicle capture particles are associated with a binding agent. In some embodiments, the binding agent binds to the vesicle to be captured from the sample. In some embodiments, the binding agent can include, without limitation, antibodies, peptides, aptamers, nucleic acids, peptide nucleic acids, polymers, molecularly imprinted polymers, molecules capable of facilitating hydrostatic interactions, and combinations thereof. In some embodiments, the binding agent includes antibodies. In some embodiments, the binding agent includes aptamers.

### First Surface

First surfaces generally refer to platform regions that can immobilize particle-vesicle complexes. As set forth in further detail herein, the methods and platforms of the present disclosure can include various first surfaces.

For instance, in some embodiments, the first surface includes a magnetized region or a region exposed to a magnetic field. In some embodiments, the region is utilized to immobilize the vesicle capture particles. In some embodiments, the region includes a magnet positioned in proximity to the first surface. In some embodiments, the magnet can include, without limitation, permanent magnets, electromagnets, soft magnets, magnetic particles associated with polymers, and combinations thereof.

In some embodiments, the first surface includes a functionalized region. In some embodiments, the functionalized region is functionalized with at least one functional group. In some embodiments, the at least one functional group is utilized to immobilize the vesicle capture particles. In some embodiments, the functional group can include, without limitation, charged groups, binding agents, functional groups capable of facilitating electrostatic interactions, and combinations thereof.

In some embodiments, the first surface includes a magnetic surface. In some embodiments, the magnetic surface includes polymers and magnetic particles associated with the polymers. In some embodiments, the magnetic surface is capable of generating heat upon exposure to AMF. In some embodiments, the first surface is in the form of the contact-free vesicle lysis systems of the present disclosure (e.g., vesicle lysis system 60 shown in FIG. 1F).

In some embodiments, the first surface includes a porous region. In some embodiments, the porous region is utilized to immobilize the vesicle capture particles through size-based separation.

### Immobilizing

In some embodiments, the methods of the present disclosure can further include a step of immobilizing particle-vesicle complexes on first surfaces of platforms. Immobilization can occur through various methods. For example, in some embodiments, the immobilizing occurs by a method that can include, without limitation, magnet-based immobilization, pelleting, centrifugation, size-based separations, filtration, inertial separations, acoustofluidic separations, material property based separations, dielectrophoretic separations, immunoaffinity-based separation, and combinations thereof.

In some embodiments, the immobilizing includes applying a magnetic field to the first surface of the platform. In some embodiments, the magnetic field immobilizes the particle-vesicle complexes on the first surface of the platform. In some embodiments, the magnetic field is applied below the first surface of the platform.

In some embodiments, the immobilizing occurs through adhesion of the particle-vesicle complexes to the first surface. In some embodiments, the adhesion includes a charged interaction between the first surface and the particle-vesicle complexes.

### Lysing

As set forth in further detail herein, the methods of the present disclosure can utilize various techniques to lyse vesicles. For instance, in some embodiments, the lysing can occur by, for example, applying heat to a platform, exposing the platform to an alternating magnetic field, applying a lysis material to the platform, applying a chemical lysis agent to the platform, freezing, mechanical perturbation, and combinations thereof.

In some embodiments, the lysing occurs by exposing the platform to an alternating magnetic field (AMF). In some embodiments, the platform is exposed to an AMF that is powered by a supply associated with the platform.

In some embodiments where the first surface includes a magnetic surface, the lysing can include, for example, applying an alternating magnetic field to the magnetic surface. In some embodiments, the alternating magnetic field heats the magnetic surface and thereby generates heat. In some embodiments, the generated heat lyses the vesicles of the particle-vesicle complexes. In some embodiments, the generated heat lyses the vesicles without direct heating or addition of lysis materials. In some embodiments, the lysing occurs through no direct interaction with the vesicle.

In some embodiments where the first surface includes a magnetic surface (e.g., a polymer and magnetic particles associated with the polymer), the lysing can include, for example, applying an alternating magnetic field to the first surface. In some embodiments, the alternating magnetic field heats the magnetic surface and thereby generates heat. In some embodiments, the generated heat lyses the vesicles of the particle-vesicle complexes. In some embodiments, the generated heat lyses the vesicles without direct heating or addition of lysis materials. In some embodiments, the lysing occurs through no direct interaction with the vesicle.

In some embodiments, the lysing occurs by applying a lysis material to the platform. In some embodiments, the lysis material can include, without limitation, a detergent, a chemical lysis buffer, a biological lysis buffer, and combinations thereof.

### Second Surface

Second surfaces generally refer to platform regions that can detect analytes. In some embodiments, the second surface is the same as the first surface. In some embodiments, the second surface is adjacent or proximal to the first surface. In some embodiments, the second surface is downstream from the first surface.

The methods and platforms of the present disclosure can include various second surfaces. For instance, in some embodiments, the second surface may include one or more analyte detecting agents. In some embodiments, the second surface may be in the form of the sensors of the present disclosure (e.g., sensor 30 shown in FIG. 1C).

In some embodiments, the second surface can include a dielectric surface and nanostructures associated with the dielectric surface. In some embodiments, the nanostructures are coupled to an analyte detecting agent. In some embodiments, the dielectric surface can include, for example, a glass surface, a plastic surface, a polymer surface, a metallic surface, a ceramic surface, and combinations thereof. In some embodiments, the dielectric surface includes a glass surface.

In some embodiments, the dielectric surface includes a metallic surface. In some embodiments, the metallic surface includes at least one metal. In some embodiments, the at least one metal can include, without limitation, gold, silver, copper, transition metals, metals, metalloids, and combinations thereof. In some embodiments, the metallic surface is composed essentially of gold.

In some embodiments, the nanostructures can include, without limitation, plasmonic nanoparticles, metal nanoparticles, magnetic nanoparticles, functionalized nanoparticles, functionalized magnetic nanoparticles, nanorods, nanospheres, nanocubes, magnetic nanorods, functionalized nanorods, functionalized magnetic nanorods, and combinations thereof. In some embodiments, the nanostructures include plasmonic nanoparticles.

In some embodiments, the nanostructures are directly associated with the dielectric surface through direct contact between the nanostructures and the dielectric surface. In some embodiments, the nanostructures are indirectly associated with the dielectric surface through indirect contact between the nanostructures and the dielectric surface. In some embodiments, the nanostructures are directly fabricated atop the dielectric surface. In some embodiments, the nanostructures are indirectly associated with the dielectric surface through the analyte detecting agent. In some embodiments, at least a portion of the analyte detecting agent is positioned between the nanostructures and the dielectric surface.

In some embodiments, the analyte detecting agent shortens upon binding to the analyte, thereby bringing the nanostructure closer to the dielectric surface, and thereby resulting in the change in the property of the second surface.

In some embodiments, the second surface is in a form of an array. In some embodiments, the array includes a plurality of different analyte detecting agents that are specific for detecting different analytes. As such, in some embodiments, the methods of the present disclosure can be utilized to detect a plurality of different analytes.

### Analyte Detecting Agents

The methods of the present disclosure can associate analytes with analyte detecting agents in various manners. For example, in some embodiments, associating the analyte with an analyte detection agent includes specifically binding the analyte detecting agent to the analyte.

The methods and platforms of the present disclosure can utilize various analyte detecting agents. For instance, in some embodiments, the analyte detecting agents can include, without limitation, aptamers, oligonucleotides, single-stranded oligonucleotides, double-stranded oligonucleotides, DNA, RNA, single stranded DNA, antibodies, peptide nucleic acids (PNAs), and combinations thereof. In some embodiments, the analyte detecting agent includes peptide nucleic acids (PNAs).

Analyte detecting agents may be associated with the platforms of the present disclosure in various manners. For instance, in some embodiments, the analyte detecting agents are directly associated with a second surface of a platform. In some embodiments, the analyte detecting agents are indirectly associated with a second surface of a platform through association with one or more nanostructures. In some embodiments, the analyte detecting agents may be immobilized on a second surface of a platform through, for example, covalent coupling, hydrostatic coupling, electrostatic coupling, and combinations thereof.

### Changes in Properties of Second Surfaces

As outlined herein, the methods of the present disclosure can rely on various changes in properties of a second surface to detect an analyte in a sample. For instance, in some embodiments, the change in property is characterized by a change in absorbance of the second surface, a shift in peak absorbance wavelength of the second surface, a shift in transmittance wavelength of the second surface, a shift in reflectance wavelength of the second surface, a shift in extinction wavelength of the second surface, a change in plasmonic field intensity of the second surface, enhanced resonance sensitivity, a color change in dark field image from the second surface, a change in an image of the second surface, a shortening of the analyte detecting agent, a change in measured light absorbance, a change in transmittance, a change in reflectance, a change in extinction, and combinations thereof. In some embodiments, the change in property is characterized by a shift in peak absorbance wavelength of the second surface.

The methods of the present disclosure can also detect a change in a property of a second surface in various manners. For example, in some embodiments, the detecting the change in property occurs by a method that can include, without limitation, visualization, microscopy, dark field microscopy, spectrometry, spectroscopy, colorimetric analysis, localized surface plasmon resonance (LSPR), nuclear magnetic resonance (NMR), surface plasmon resonance, electrochemistry, and combinations thereof. In some embodiments, the detecting the change in property includes visualizing a color or image change of the second surface on a simple dark field image.

### Correlation of a Change in Property to an Analyte Characteristic

As set forth in further detail herein, the methods of the present disclosure can utilize various techniques to correlate a change in property of a second surface to a characteristic of an analyte. For instance, in some embodiments, the correlating occurs in a quantitative, semi quantitative, or qualitative manner.

Additionally, the methods of the present disclosure can be utilized to determine various characteristics of an analyte. For example, in some embodiments, the characteristic of the analyte can include, without limitation, the identity of the analyte, the presence of the analyte, the absence of the analyte, the concentration of the analyte, the quantity of the analyte, and combinations thereof.

### Platforms

As detailed herein, the methods of the present disclosure can utilize various platforms for the detection of analytes. For instance, in some embodiments, the platform includes a channel. In some embodiments, the channel can include, without limitation, a microchannel, a fluid channel, and combinations thereof.

In some embodiments, the channel includes an inlet section for receiving the sample and a mixing region for mixing the sample with the vesicle capture particles to form the particle-vesicle complexes. In some embodiments, the mixing region is downstream the first inlet.

In some embodiments, the platform includes the first surface for capturing the particle-vesicle complexes. In some embodiments, the first surface is downstream the mixing region. In some embodiments, the platform includes the second surface for detecting the analyte.

In some embodiments, the platform further includes a magnet in proximity to the first surface. In some embodiments, the inlet section includes a first inlet and a second inlet converging into the mixing region. In some embodiments, the first sample is introduced into the channel through the first inlet and the vesicle capture particles are introduced into the channel through the second inlet.

In some embodiments, the channel includes channels with diameters of less than 1 mm. In some embodiments, the channel includes a portion with a configuration that can include, without limitation, a jagged configuration, a serpentine configuration, a hexagonal configuration, a spiral-shaped configuration, linear configuration, H-configuration, and combinations thereof.

In some embodiments, the channel includes a portion with a spiral shaped configuration. In some embodiments, the channel includes a portion with capillary pump.

In some embodiments, the platform is in the form of a microchannel. In some embodiments, the platform is in the form of the analyte detection platforms of the present disclosure (e.g., analyte detection platform 20 shown in FIG. 1A).

### Embodiments and Applications

As set forth in further detail herein, the analyte detection methods of the present disclosure can have numerous embodiments and applications. For instance, in some embodiments, the analyte detection methods of the present disclosure occur without amplification, replication, growth, or culture of the analyte. In some embodiments, the analyte detection methods of the present disclosure occur without amplification, replication, growth, or culture of the vesicles.

In some embodiments, the analyte detection methods of the present disclosure utilized for the characterization, detection, or quantification of a plurality of different analytes. In some embodiments, the analyte detection methods of the present disclosure are utilized for characterization of an infection, cancer, or chronic illness. In some embodiments, the infection may be, for example, bacterial infections, viral infections, polymicrobial infections, and combinations thereof.

### Analyte Detection Platform

As set forth in further detail herein, an aspect of the present disclosure relates to a platform for analyte detection in a sample. In some embodiments, the platform can include an inlet region for receiving a sample, a mixing region for mixing the sample, a capturing region including a first surface for capturing one or more components of the sample, where the first surface is downstream the mixing region, and a sensing region that includes a second surface for detecting an analyte from the sample. In some embodiments, the second surface includes an analyte detecting agent.

The analyte detection platforms of the present disclosure can include various configurations. For instance, in some embodiments, the analyte detection platforms of the present disclosure may be in the form of analyte detection platform 20 shown in FIG. 1A1. As described in more detail herein, the analyte detection platforms of the present disclosure can include numerous additional embodiments and variations.

### Inlet Region

As set forth in detail herein, the platforms of the present disclosure can include various inlet regions with various configurations. For example, in some embodiments, the inlet region includes a first inlet and a second inlet converging into the mixing region. In some embodiments, the inlet region includes single inlet region converging into the mixing region.

### Capturing Region

As set forth in further detail herein, the platforms of the present disclosure can include various capturing regions and first surface configurations. For instance, in some embodiments, the capturing region further includes a magnet positioned in proximity to the first surface. In some embodiments, the magnet can include, without limitation, permanent magnets, electromagnets, soft magnets, alternating current magnets, and combinations thereof. In some embodiments, the magnet is heated by an alternating magnetic field. In some embodiments, the capturing region includes a magnetic surface. In some embodiments, the magnetic surface generates heat upon exposure to AMF.

In some embodiments, the capturing region includes a magnetic surface. In some embodiments, the magnetic surface includes a polymer and magnetic particles associated with the polymer. In some embodiments, the capturing region includes first surfaces that have been previously described in detail in this Application. In some embodiments, capturing region is in the form of the contact-free vesicle lysis systems of the present disclosure (e.g., vesicle lysis system 60 shown in FIG. 1F).

### Sensing Region

As set forth in further detail below, the platforms of the present disclosure can include various sensing regions and second surface configurations. For example, in some embodiments, the second surface includes second surfaces that have been previously described in detail in this Application. In some embodiments, the second surface includes a dielectric surface and nanostructures associated with the dielectric surface. In some embodiments, the nanostructures are coupled to the analyte detecting agent.

In some embodiments, the dielectric surface includes, for example, a glass surface, a plastic surface, a polymer surface, a transparent surface, a metallic surface, a ceramic surface, and combinations thereof. In some embodiments, the dielectric surface includes a glass surface. In some embodiments, the dielectric surface includes a metallic surface. In some embodiments, the metallic surface includes at least one metal. In some embodiments, the at least one metal can include, without limitation, gold, platinum, silver, copper, transition metals, metals, metalloids, and combinations thereof. In some embodiments, the metallic surface is composed essentially of gold.

In some embodiments, the nanostructures can include, without limitation, plasmonic nanoparticles, metal nanoparticles, magnetic nanoparticles, functionalized nanoparticles, functionalized magnetic nanoparticles, nanorods, nanospheres, nanocubes, magnetic nanorods, functionalized nanorods, functionalized magnetic nanorods, and combinations thereof. In some embodiments, the nanostructures include plasmonic nanoparticles.

In some embodiments, the nanostructures are directly associated with the dielectric surface through direct contact between the nanostructures and the dielectric surface. In some embodiments, the nanostructures are indirectly associated with the dielectric surface through indirect contact between the nanostructures and the dielectric surface. In some embodiments, the nanostructures are indirectly associated with the dielectric surface through the analyte detecting agent. In some embodiments, at least a portion of the analyte detecting agent is positioned between the nanostructures and the dielectric surface. In some embodiments, the analyte detecting agent shortens upon binding to the analyte, thereby bringing the nanostructure closer to the dielectric surface.

In some embodiments, the second surface is in a form of an array. In some embodiments, the array includes a plurality of different analyte detecting agents that are specific for detecting different analytes.

In some embodiments, the second surface is the same as the first surface. In some embodiments, the second surface is adjacent or proximal to the first surface. In some embodiments, the second surface is downstream from the first surface.

In some embodiments, the second surface may be in the form of the sensors of the present disclosure (e.g., sensor 30 shown in FIG. 1C).

### Analyte Detecting Agent

As detailed herein, the platforms of the present disclosure can include various analyte detection agents. For example, in some embodiments, the analyte detecting agent specifically binds to an analyte. In some embodiments, the analyte can include, without limitation, nucleotides, RNA, DNA, ribosomal RNA (rRNA), messenger RNA (mRNA), microDNA, microRNA, extrachromosomal circular DNA (eccDNA), cell free DNA (cfDNA), circulating tumor DNA (ctDNA), small molecules, proteins, mutated versions thereof, and combinations thereof.

In some embodiments, the analyte detecting agent can include, without limitation, aptamers, oligonucleotides, single-stranded oligonucleotides, double-stranded oligonucleotides, DNA, RNA, single stranded DNA, antibodies, peptide nucleic acids (PNAs), selective polymers, and combinations thereof. In some embodiments, the analyte detecting agent includes peptide nucleic acids (PNAs).

Analyte detecting agents may be associated with second surfaces of platforms in various manners. For instance, in some embodiments, the analyte detecting agents are directly associated with the second surface of a platform. In some embodiments, the analyte detecting agents are indirectly associated with the second surface of a platform through association with one or more nanostructures. In some embodiments, the analyte detecting agents may be immobilized on a second surface of a platform through, for example, covalent coupling, hydrostatic coupling, electrostatic coupling, and combinations thereof.

### Platform Configuration

As set forth in further detail herein, the platforms of the present disclosure can have numerous configurations. For example, in some embodiments, the platform includes channels with diameters of less than 1 mm. In some embodiments, the platform includes a configuration that can include, without limitation, a jagged configuration, a serpentine configuration, a hexagonal configuration, a spiral-shaped configuration, linear configuration, H-configuration, and combinations thereof.

In some embodiments, the platform includes a spiral shaped configuration. In some embodiments, the platform is in the form of a channel. In some embodiments, the platform is in the form of a microchannel.

### Sensors

Another aspect of the present disclosure pertains to sensors used for analyte detection. In some embodiments, the sensor includes a surface for detecting an analyte from a sample. In some embodiments, the surface includes a dielectric surface and nanostructures randomly oriented on the dielectric surface. In some embodiments, the nanostructures are coupled to an analyte detecting agent. In some embodiments, the sensor is a plasmonic sensor.

The sensors of the present disclosure can include various configurations. For instance, in some embodiments, the sensors of the present disclosure may be in the form of sensor 30 shown in FIG. 1C. As described in more detail herein, the sensors of the present disclosure can include numerous additional embodiments and variations.

### Dielectric Surfaces

As set forth in further detail herein, the sensors of the present disclosure can utilize various dielectric surfaces. For instance, in some embodiments, the dielectric surface includes, for example, a glass surface, a plastic surface, a polymer surface, a metallic surface, a ceramic surface, a transparent surface, and combinations thereof. In some embodiments, the dielectric surface includes a glass surface.

In some embodiments, the dielectric surface includes a metallic surface. In some embodiments the metallic surface includes at least one metal. In some embodiments, the at least one metal can include, without limitation, gold, platinum, silver, copper, transition metals, metals, metalloids, and combinations thereof. In some embodiments, the metallic surface is composed essentially of gold.

### Nanostructures

As detailed herein, the sensors of the present disclosure can include various nanostructures. For example, in some embodiments, the nanostructures can include, without limitation, plasmonic nanoparticles, metal nanoparticles, magnetic nanoparticles, functionalized nanoparticles, functionalized magnetic nanoparticles, gold nanoparticles, nanorods, nanospheres, nanocubes, magnetic nanorods, functionalized nanorods, functionalized magnetic nanorods, and combinations thereof. In some embodiments, the nanostructures include plasmonic nanoparticles.

In some embodiments, the nanostructures include at least one metal. In some embodiments, the at least one metal can include, without limitation, gold, platinum, silver, copper, transition metals, metals, metalloids, and combinations thereof.

In some embodiments, the nanostructures are directly associated with the dielectric surface through direct contact between the nanostructures and the dielectric surface. In some embodiments, the nanostructures are indirectly associated with the dielectric surface through indirect contact between the nanostructures and the dielectric surface. In some embodiments, the nanostructures are dispersed using fluid flow onto the dielectric surface.

In some embodiments, the nanostructures are indirectly associated with the dielectric surface through the analyte detecting agent. In some embodiments, the analyte detecting agent is positioned between the nanostructures and the dielectric surface. In some embodiments, the analyte detecting agent shortens upon binding to the analyte, thereby bringing the nanostructure closer to the dielectric surface.

In some embodiments, the surface is in a form of an array. In some embodiments, the array includes a plurality of different analyte detecting agents that are specific for different analytes. In some embodiments, the plurality of different analyte detecting agents are coupled to the same or different nanostructures. In some embodiments, the nanostructures are covalently bound to the dielectric surface. In some embodiments, the nanostructures are electrostatically bound to the dielectric surface.

In some embodiments, the nanostructures include diameters ranging from about 30 nm to about 500 nm. In some embodiments, the nanostructures include diameters ranging from about 30 nm to about 100 nm. In some embodiments, the nanostructures include diameters of at least about 30 nm. In some embodiments, the nanostructures include diameters of at least about 100 nm. In some embodiments, the nanostructures include diameters of less than about 100 nm.

### Random Orientation

As set forth in further detail herein, the nanostructures of the sensors of the present disclosure can have a random orientation on dielectric surfaces. For instance, in some embodiments, the nanostructures are randomly dispersed on the dielectric surface. In some embodiments, the nanostructures are randomly oriented such that their long axes are not all in the same direction. In some embodiments, the nanostructures are randomly oriented such that their long axes are all in the same direction.

### Analyte Detecting Agents

As set forth in further detail herein, the sensors of the present disclosure can include various analyte detecting agents. For example, in some embodiments, the analyte detecting agent specifically binds to an analyte. In some embodiments, the analyte can include, without limitation, nucleotides, RNA, DNA, ribosomal RNA (rRNA), messenger RNA (mRNA), microDNA, microRNA, extrachromosomal circular DNA (eccDNA), cell free DNA (cfDNA), circulating tumor DNA (ctDNA), small molecules, proteins, mutated versions thereof, and combinations thereof. In some embodiments, the analyte includes cell free DNA (cfDNA). In some embodiments, the analyte includes nucleotides derived from lysed cells.

In some embodiments, the analyte detecting agent can include, without limitation, aptamers, oligonucleotides, single-stranded oligonucleotides, double-stranded oligonucleotides, DNA, RNA, single stranded DNA, antibodies, peptide nucleic acids (PNAs), polymers, and combinations thereof. In some embodiments, the analyte detecting agent includes peptide nucleic acids (PNAs).

Nanostructures may be coupled to analyte detecting agents in various manners. For instance, in some embodiments, the analyte detecting agent is immobilized on the nanostructures through covalent coupling. In some embodiments, the analyte detecting agent is immobilized on the nanostructures through electrostatic coupling.

### Configuration

As set forth in further detail herein, the sensors of the present disclosure can have numerous configurations. For example, in some embodiments, the sensor includes channels with diameters of less than 1 mm. In some embodiments, the sensor has a configuration that can include, without limitation, a jagged configuration, a serpentine configuration, a hexagonal configuration, a spiral-shaped configuration, linear configuration, H-configuration, and combinations thereof.

In some embodiments, the sensor includes a spiral shaped configuration. In some embodiments, the sensor is in the form of a microchannel. In some embodiments, the sensor is in the form of a chamber. In some embodiments, the sensor can have 350 µm × 750 µm ovals in 10 × 10 arrays.

The sensors of the present disclosure may be components of various devices. For instance, in some embodiments, the sensors of the present disclosure may be components of the analyte detection platforms of the present disclosure.

### Sensing

As set forth in further detail herein, another aspect of the present disclosure pertains to sensing. For example, in some embodiments, the present disclosure pertains to a method of detecting an analyte from a sample through one or more of the following steps: (a) flowing the sample through a sensor; and (b) detecting the analyte. In some embodiments, the analyte detection includes detecting a change in property of a sensor surface and correlating the change in property of the surface to a characteristic of the analyte. In some embodiments, the sensing is plasmonic sensing.

In some embodiments, the sensor surface includes a dielectric surface and nanostructures randomly oriented on the dielectric surface. In some embodiments, the nanostructures are coupled to an analyte detecting agent. In some embodiments, the sensor includes the sensors of the present disclosure, including the dielectric surfaces, nanostructures, and analyte detecting agents described previously in this Application for such sensors.

### Samples

As set forth in further detail herein, analytes can be detected from various types of samples. For example, in some embodiments, the sample can include, without limitation, a biological sample obtained from a subject, an environmental sample obtained from an environment, a swab sample, and combinations thereof. In some embodiments, the sample includes a biological sample obtained from a subject. In some embodiments, the biological sample can include, without limitation, a blood sample, a tissue sample, a urine sample, a saliva sample, a sputum sample, a swab sample, a swab sample put into a carrier solution, a processed blood sample, and combinations thereof.

In some embodiments, the sample includes an environmental sample. In some embodiments, the environmental sample can include, without limitation, a food sample, a water sample, a swab sample, a swab sample put into a carrier solution, a surface swab sample, a passive material sample put into a carrier solution, and combinations thereof.

### Flowing the Samples

As outlined in further detail herein, the methods of the present disclosure can utilize various methods of flowing the sample through a sensor. For example, in some embodiments, the flowing includes flowing the sample over the sensor.

In some embodiments, the flowing occurs through a method that can include, without limitation, pumping, mechanical pumping, electrical pumping, syringe-facilitated flow, pipette-facilitated flow, capillary flow, peristaltic flow, pressure-driven flow, and combinations thereof.

### Analytes

As set forth in further detail below, various analytes can be detected via the methods of the present disclosure. For example, in some embodiments, the analyte can include, without limitation, nucleotides, oligonucleotides, wild-type nucleotides, mutated nucleotides, double-stranded nucleotides, RNA, DNA, ribosomal RNA (rRNA), messenger RNA (mRNA), microDNA, microRNA, extrachromosomal circular DNA (eccDNA), cell free DNA (cfDNA), circulating tumor DNA (ctDNA), small molecules, proteins, mutated versions thereof, and combinations thereof.

In some embodiments, the analyte includes RNA. In some embodiments, the analyte includes cell free DNA (cfDNA). In some embodiments, the analyte includes nucleotides derived from lysed cells. In some embodiments, the analyte includes mutated nucleotides.

### Surfaces

The sensors that are utilized for the methods of the present disclosure can include various surfaces. For instance, in some embodiments, the surface includes a dielectric surface. In some embodiments, the dielectric surface can include, for example, a glass surface, a metallic surface, a plastic surface, a polymer surface, a ceramic surface, and combinations thereof. In some embodiments, the dielectric surface includes a glass surface.

In some embodiments, the dielectric surface includes a metallic surface. In some embodiments, the metallic surface includes at least one metal. In some embodiments, the at least one metal can include, without limitation, gold, platinum, silver, copper, transition metals, metals, metalloids, and combinations thereof. In some embodiments, the metallic surface is composed essentially of gold.

In some embodiments, the nanostructures can include, without limitation, plasmonic nanoparticles, metal nanoparticles, magnetic nanoparticles, functionalized nanoparticles, functionalized magnetic nanoparticles, nanorods, nanospheres, nanocubes, magnetic nanorods, functionalized nanorods, functionalized magnetic nanorods, and combinations thereof.

In some embodiments, the nanostructures are directly associated with the dielectric surface through direct contact between the nanostructures and the dielectric surface. In some embodiments, the nanostructures are indirectly associated with the dielectric surface through indirect contact between the nanostructures and the dielectric surface. In some embodiments, the nanostructures are indirectly associated with the nanostructures through the analyte detecting agent. In some embodiments, the analyte detecting agent is positioned between the nanostructures and the dielectric surface.

In some embodiments, the analyte detecting agent shortens upon binding to the analyte, thereby bringing the nanostructure closer to the dielectric surface, and thereby resulting in the change in the property of the surface. In some embodiments, the surface is in a form of an array. In some embodiments, the array includes a plurality of different analyte detecting agents that are specific for different analytes. As such, in some embodiments, the method is utilized to detect a plurality of different analytes.

### Analyte Detecting Agents

As detailed herein, the sensors that are utilized in accordance with the methods of the present disclosure can include various analyte detecting agents. For instance, in some embodiments, the analyte detecting agent specifically binds to the analyte. In some embodiments, the analyte detecting agent can include, without limitation, aptamers, oligonucleotides, single-stranded oligonucleotides, double-stranded oligonucleotides, DNA, RNA, single stranded DNA, antibodies, peptide nucleic acids (PNAs), and combinations thereof. In some embodiments, the analyte detecting agent includes peptide nucleic acids (PNAs).

Nanostructures may be coupled to analyte detecting agents in various manners. For instance, in some embodiments, the analyte detecting agent is immobilized on the nanostructures through covalent coupling. In some embodiments, the analyte detecting agent is immobilized on the nanostructures through electrostatic coupling.

### Detecting a Change in Property

As outlined herein, the methods of the present disclosure can utilize various changes in properties of a surface to detect an analyte in a sample. For instance, in some embodiments, the change in property is characterized by a change in absorbance of the surface, a shift in peak absorbance wavelength of the surface, a change in plasmonic field intensity of the surface, enhanced resonance sensitivity, a color change in dark field image from the surface, a change in an image of the surface, a shortening of the analyte detecting agent, a change in measured light absorbance, a change in transmittance, a change in reflectance, a change in extinction, and combinations thereof. In some embodiments, the change in property is characterized by a shift in peak absorbance of the surface.

The methods of the present disclosure can also detect a change in a property of a surface in various manners. For example, in some embodiments, the detecting the change in property occurs by a method that can include, without limitation, visualization, microscopy, dark field microscopy, spectrometry, spectroscopy, colorimetric analysis, localized surface plasmon resonance (LSPR), surface plasmon resonance, electrochemistry, nuclear magnetic resonance (NMR), and combinations thereof. In some embodiments, the detecting includes visualizing a color or image change of the surface on a simple dark field image.

### Correlation of a Change in Property to an Analyte Characteristic

As set forth in further detail herein, the methods of the present disclosure can utilize various techniques to correlate a change in property of a surface to a characteristic of an analyte. For instance, in some embodiments, the correlation occurs in a quantitative, semiquantiative, or qualitative manner.

Additionally, the methods of the present disclosure can be utilized to determine various characteristics of an analyte. For example, in some embodiments, the characteristic of the analyte can include, without limitation, the identity of the analyte, the presence of the analyte, the absence of the analyte, the concentration of the analyte, the quantity of the analyte, and combinations thereof.

### Embodiments and Applications

As detailed herein, the methods of the present disclosure can have various embodiments and applications. For example, in some embodiments, the method occurs without amplification, replication, growth, or culture of the analyte. In some embodiments, the method is utilized for the characterization of a plurality of different analytes.

### Contact-Free Vesicle Lysis

As described in further detail herein, embodiments of the present disclosure relate to contract-free vesicle lysis methods. For example, in some embodiments, the present disclosure pertains to methods of lysing vesicles in a sample through one or more of the following steps: (a) flowing the sample through a platform, where vesicle capture particles bind to the vesicles in the sample to form particle-vesicle complexes, and where the particle-vesicle complexes become immobilized on a surface of the platform; and (b) lysing the vesicles of the particle-vesicle complexes. In some embodiments, the methods of the present disclosure can also include a step of collecting an analyte released from the lysed vesicles. In some embodiments, the collecting includes flowing the released analyte from the surface into a container.

### Platform Surfaces

The methods of the present disclosure can utilize various platform surfaces. For instance, in some embodiments, the platform surface includes a magnetic surface. In some embodiments, the magnetic surface includes a polymer and magnetic particles associated with the polymer. In some embodiments, the magnetic surface is capable of generating heat upon exposure to an alternating magnetic field (AMF).

Magnetic surfaces that include polymers and magnetic materials may be in various forms. For instance, in some embodiments, the magnetic surface is in the form of a polymer composite. In some embodiments, the magnetic surfaces is in the form of a polymer matrix. In some embodiments, the magnetic particles are imbedded with the polymer.

The magnetic surfaces of the present disclosure can include various polymers. For example, in some embodiments, the polymer can include, without limitation, polydimethylsiloxane (PMDS), polymethylmethacrylate (PMMA), polyethylene glycol (PEG), polyvinylidene fluoride (PVDF), and combinations thereof. In some embodiments, the polymer includes polydimethylsiloxane (PDMS).

The magnetic surfaces of the present disclosure can also include various magnetic particles. For instance, in some embodiments, the magnetic particles can include, without limitation, single-domain magnetic particles, multi-domain magnetic particles, magnetic nanoparticles, iron oxide particles, and combinations thereof.

In addition to magnetic surfaces, the platform surfaces of the present disclosure can also include additional components. For example, in some embodiments, the surface includes a magnet. In some embodiments, the magnet is utilized to immobilize the vesicle capture particles. In some embodiments, the magnet includes a magnet positioned in proximity to the surface. In some embodiments, the magnet can include, without limitation, permanent magnets, electromagnets, soft magnets, alternating current magnets, and combinations thereof.

### Samples

As discussed in further detail herein, the methods of the present disclosure can detect analytes in various types of samples. For instance, in some embodiments, the sample can include, without limitation, a biological sample obtained from a subject, an environmental sample obtained from an environment, and combinations thereof.

In some embodiments, the sample includes a biological sample obtained from a subject. In some embodiments, the biological sample can include, without limitation, a blood sample, a tissue sample, a urine sample, a saliva sample, a sputum sample, a swab sample, a swab sample put into a carrier solution, a processed blood sample, and combinations thereof.

In some embodiments, the sample includes an environmental sample. In some embodiments, the environmental sample can include, without limitation, a food sample, a water sample, a swab sample, a swab sample put into a carrier solution, a surface swab sample, a passive material sample put into a carrier solution, and combinations thereof.

### Flowing the Samples

As outlined herein, the methods of the present disclosure can utilize various manners of flowing the sample through the platforms of the present disclosure. For instance, in some embodiments, the flowing occurs through a method that can include, without limitation, pumping, mechanical pumping, electrical pumping, syringe-facilitated flow, pipette-facilitated flow, capillary flow, peristaltic flow, pressure-driven flow, and combinations thereof.

In some embodiments, the flowing includes flowing the sample through the platform along with the vesicle capture particles. In some embodiments, the sample is co-introduced into the platform along with the vesicle capture particles. In some embodiments, the sample is pre-incubated with the vesicle captures particles prior to co-introduction into the platform. In some embodiments, the flowing includes flowing the sample through the platform while the vesicle capture particles are immobilized on a surface of the platform. In some embodiments, the method further includes a step of immobilizing the vesicle capture particles on the surface prior to the flowing step.

### Vesicles

As detailed herein, the methods of the present disclosure can be utilized to lyse various vesicles. For example, in some embodiments, the vesicles can include, without limitation, viruses, bacteria, yeast, fungi, prokaryotic cells, eukaryotic cells, extracellular vesicles, and combinations thereof. In some embodiments, the vesicles include bacteria.

In some embodiments, the vesicles include viruses. In some embodiments, the vesicles include severe acute respiratory syndrome coronavirus 2 (SARS-CoV-2). In some embodiments, the vesicles include Human Papilloma Virus (HPV).

In some embodiments, the vesicles include eukaryotic cells. In some embodiments, the eukaryotic cells include cancer cells.

In some embodiments, the vesicles include extracellular vesicles. In some embodiments, the extracellular vesicles include exosomes.

### Vesicle Capture Particles

As detailed herein, the methods of the present disclosure can utilize numerous vesicle capture particles. For example, in some embodiments, the vesicle capture particles can include, without limitation, metal particles, magnetic particles, polymer-based particles, gelled particles, and combinations thereof.

In some embodiments, the vesicle capture particles include magnetic particles. In some embodiments, the vesicle capture particles are associated with a binding agent. In some embodiments, the binding agent binds to the vesicle to be captured from the sample. In some embodiments, the binding agent can include, without limitation, antibodies, peptides, aptamers, oligonucleotides, polymers, molecularly imprinted polymers, and combinations thereof. In some embodiments, the binding agent includes antibodies.

### Immobilizing

In some embodiments, the methods of the present disclosure include a step of immobilizing particle-vesicle complexes on a surface of a platform. As detailed herein, various methods may be utilized to immobilize particle-vesicle complexes onto surfaces. In some embodiments, the immobilizing occurs by a method that can include, without limitation, magnet-based immobilization, pelleting, centrifugation, size-based separations, filtration, inertial separations, acoustofluidic separations, material property based separations,. dielectrophoretic separations, immunoaffinity-based separation, and combinations thereof.

In some embodiments, the immobilizing includes applying a magnetic field to a surface of a platform. In some embodiments, the magnetic field immobilizes the particle-vesicle complexes on the surface of the platform.

In some embodiments, the immobilizing occurs through adhesion of the particle-vesicle complexes to the surface. In some embodiments, the adhesion includes a charged interaction between the surface and the particle-vesicle complexes.

### Lysing

As disclosed in further detail herein, the methods of the present disclosure can utilize various lysing methods and techniques to lyse vesicles. For instance, in some embodiments, the lysing occurs through no direct interaction with the vesicle. In some embodiments, the lysing includes exposing the surface to an alternating magnetic field (AMF). In some embodiments, the AMF is powered by a supply associated with the platform.

In some embodiments, the AMF heats the surface. For example, in some embodiments, the AMF heats a magnetic surface of the surface and thereby generates heat. In some embodiments, the generated heat lyses the vesicles of the particle-vesicle complexes. In some embodiments, the generated heat lyses the vesicles without direct heating or addition of lysis materials.

### Analyte Release and Collection

As outlined herein, the methods of the present disclosure can include additional steps. For example, in some embodiments, the methods further include a step of collecting an analyte released from the lysed vesicles. In some embodiments, the collecting includes flowing the released analyte from the surface into a container.

In some embodiments, the analyte can include, without limitation, nucleotides, RNA, DNA, ribosomal RNA (rRNA), messenger RNA (mRNA), microDNA, microRNA, extrachromosomal circular DNA (eccDNA), circulating tumor DNA (ctDNA), small molecules, proteins, mutated versions thereof, and combinations thereof. In some embodiments, the analyte includes DNA.

In some embodiments, the methods of the present disclosure further includes analyzing the collected analyte. In some embodiments, the analyzing includes identifying the analyte. In some embodiments, the identifying occurs by a method that can include, without limitation, chemical analysis, sequencing, amplification, mass spectroscopy, sensing, plasmonic sensing, and combinations thereof.

### Contact-Free Vesicle Lysis Systems

As outlined in further detail below, various aspects of the present disclosure pertain to contact-free vesicle lysis systems. For instance, in some embodiments, the present disclosure pertains to a vesicle lysis platform that includes a surface. In some embodiments, the surface is a magnetic surface. In some embodiments, the surface includes a magnetic surface. In some embodiments, the magnetic surface includes a polymer and magnetic particles associated with the polymer. In some embodiments, the surface is capable of generating heat upon exposure to AMF. In some embodiments, the magnetic surface is capable of generating heat upon exposure to AMF.

The vesicle lysis platforms of the present disclosure can include various configurations. For instance, in some embodiments, the vesicle lysis platforms of the present disclosure may be in the form of vesicle lysis platform 60 shown in FIG. 1F. As described in more detail herein, the vesicle lysis platforms of the present disclosure can include numerous additional embodiments and variations.

The vesicle lysis platforms of the present disclosure can include various platform surfaces. For instance, in some embodiments, the platform surface is a magnetic surface. In some embodiments, the platform surface includes a magnetic surface. In some embodiments, the magnetic surface includes a polymer and magnetic particles associated with the polymer.

### Magnetic Surfaces

In embodiments where the platform surface includes a magnetic surface, the magnetic surfaces of the vesicle lysis platforms may be in various forms. For instance, in some embodiments, the magnetic surface is in the form of a polymer composite. In some embodiments, the magnetic surface is in the form of a polymer matrix. In some embodiments, the magnetic particles are imbedded with the polymer.

The magnetic surfaces of the present disclosure can include various polymers. For example, in some embodiments, the polymer can include, without limitation, polydimethylsiloxane (PMDS), polymethylmethacrylate (PMMA), polyethylene glycol (PEG), polyvinylidene fluoride (PVDF), and combinations thereof. In some embodiments, the polymer includes polydimethylsiloxane (PDMS).

The magnetic surfaces of the present disclosure can also include various magnetic particles. For instance, in some embodiments, the magnetic particles can include, without limitation, single-domain magnetic particles, multi-domain magnetic particles, magnetic nanoparticles, iron oxide particles, and combinations thereof.

In addition to magnetic surfaces, the platform surfaces of the present disclosure can also include additional components. For example, in some embodiments, the surface includes a magnet. In some embodiments, the magnet is utilized to immobilize the vesicle capture particles. In some embodiments, the magnet includes a magnet positioned in proximity to the surface. In some embodiments, the magnet can include, without limitation, permanent magnets, electromagnets, soft magnets, alternating current magnets, and combinations thereof.

### Applications and Advantages

The present disclosure can have various advantages. For instance, in some embodiments, the systems and methods of the present disclosure have at least the following valuable features: (1) providing fast processing times; (2) providing flexible detection systems; (3) allowing for simpler designs as opposed to systems and methods currently available; and (4) providing clinically relevant molecular information. As such, as described in more detail in the examples herein, the systems and methods of the present disclosure can be utilized in various manners and for various purposes.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Integrated Microsystem for On-Chip Bacterial Capture and Molecular Profiling

This Example describes an integrated microsystem for on-chip bacterial capture and molecular profiling according to aspects of the present disclosure.

### Example 1.1. Key Specifications and Preliminary Data

As an alternative to existing time-consuming, culture-based diagnostic methods for organism detection (i.e. blood culture), Applicants present a coupled micro-scale system for the enrichment and detection of bacteria from a whole blood sample that aims to meet the specifications outline herein. In Applicants’ preliminary work, Applicants demonstrate micro-scale immunomagnetic bacterial enrichment from whole blood, and evaluate the feasibility of a novel downstream nanoplasmonic sensing platform for the detection of bacterial nucleic acids from lysed captured cells.

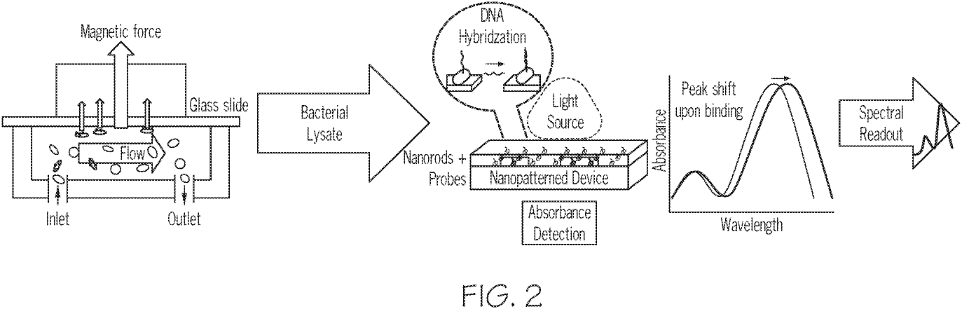

Applicants’ microscale system relies on an external magnetic field to retain bacteria bound to magnetic nanoparticles (MNPs) in the microchannel, while removing unwanted blood components, which limit detection sensitivity. Applicants’ nano-scale sensing platform relies on principles of localized surface plasmon resonance (LSPR) to detect changes in absorbance spectra in the sample. Specifically, Applicants’ device employs gold nanorods functionalized with peptide nucleic acid (PNA) probes complimentary to the sequence of interest. Following DNA hybridization to the target sequence, Applicants can observe a shift in the resonant peak (FIG. 2).

Based on previous work, Applicants employed a hexagonal-shaped microchannel for bacterial enrichment, and exposed the microchannel to an optimized external magnetic field. To prepare samples for processing, Staphylococcus aureus cells were spiked into whole blood and incubated for 1 h with 150 nm magnetic nanoparticles that were functionalized with polyclonal anti-Staphylococcus aureus antibodies. Samples were then processed on Applicants’ micro-scale enrichment system at 5 mL/h. Next, Applicants’ nanoplasmonic detection platform employed gold nanorods functionalized with peptide nucleic acid (PNA) probes complimentary to the 16s rRNA gene sequence – a region of the bacterial genome that is highly conserved between different species. The sensor was fabricated through microfluidic conjugation and assembly of gold nanorods onto a glass slide and read out using a microscope-coupled spectrometer. Applicants evaluated the efficacy of this detection approach using heat-lysed Staphylococcus aureus at varying cell concentrations.

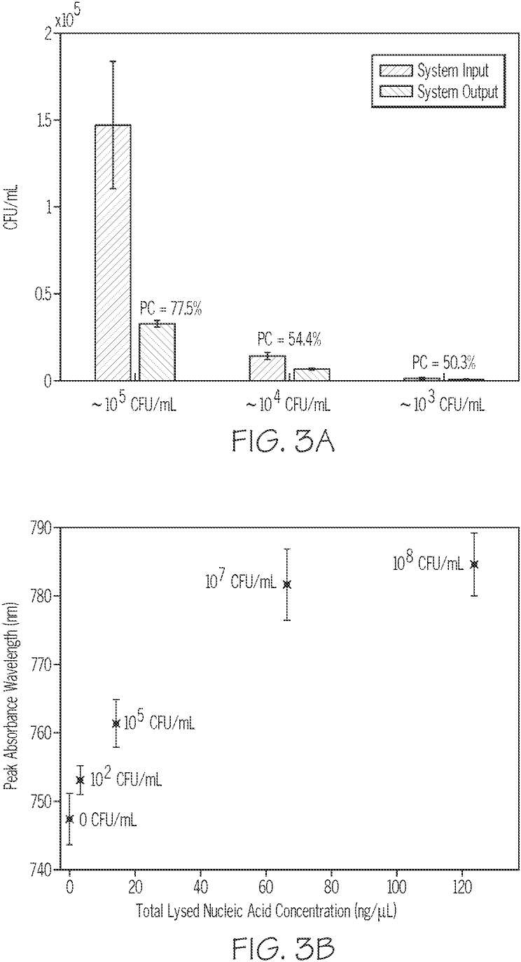

Using this system, Applicants demonstrated successful isolation of Staphylococcus aureus from non-diluted whole blood, with capture rates ranging from 50.3% ± 0.8% to 77.5% ± 1.4% (SEM) at bacterial loads on the order of 10³ cells/mL and 10⁵ cells/mL, respectively (FIG. 3A). Following cell lysis of S. aureus and exposure to Applicants’ nanoplasmonic sensing platform, Applicants observed red-shifts in peak absorbance wavelength ranging from 5.7 ± 3.8 nm to 37.3 ± 3.4 nm (SEM) relative to the mean baseline wavelength at bacterial loads on the order of 10² cells/mL and 10⁸ cells/mL, respectively (FIG. 3B). These results suggest successful hybridization of bacterial nucleic acids to PNA probes. Further, preliminary data suggest a limit of detection as low as 1000 CFU/mL using Applicants’ coupled bacterial capture and detection analysis platform.

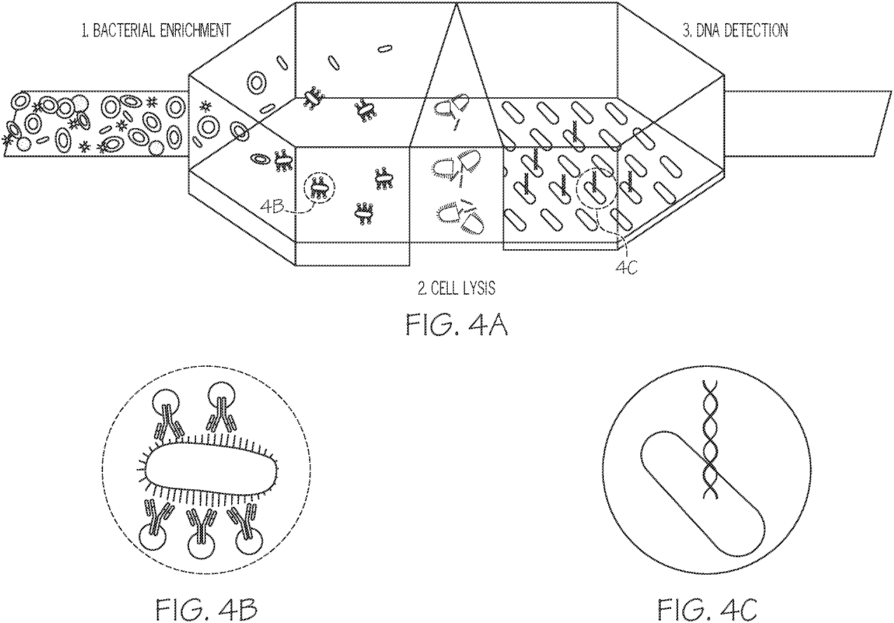

This bacterial capture and detection system has the potential to dramatically shorten time-to diagnosis, which is an important factor to improving patient outcomes. To the best of Applicants’ knowledge, this is the first Example to couple micro-scale bacterial enrichment from whole blood to plasmonic sensing. Moving forward from this initial work, Applicants aim to: (1) optimize bacterial isolation from whole blood to increase system sensitivity; and (2) integrate sample incubation, bacterial capture, and bacterial DNA detection on a single micro-chip (FIGS. 4A-C). Methods and data in support of these two objectives are specified in detail below.

### Example 1.2. Abstract

Applicants report on a high-throughput, integrated microsystem that couples immunomagnetic bacterial enrichment to nanoplasmonic molecular profiling, enabling the characterization of bacterial samples in 30 minutes. First, a bacterial sample is combined with magnetic nanoparticles (MNPs) that are functionalized with antibodies targeting bacterial surface proteins. Using Applicants’ microsystem, all sample mixing and incubation occurs entirely on-chip, minimizing required sample handling and total-analytical time. Immunomagnetic bacterial capture efficiency averaged 68.3% (± 4.9% SEM) and 41.6% (± 1.6% SEM) for S. aureus and P. aeruginosa, respectively. Following capture, bacteria are thermally lysed, and Applicants’ nanoplasmonic sensor is exposed to the bacterial lysate. Applicants’ nanoplasmonic sensing platform is composed of gold nanoparticles functionalized with peptide nucleic acid probes complimentary to species-specific nucleic acid sequences. Following hybridization of bacterial nucleic acids to the PNA-nanosensor complex, a red-shift in peak absorbance wavelength is observed. Applicants demonstrate species-specific characterization of E. coli, P. aeruginosa, and S. aureus lysate with shifts in peak absorbance wavelength up to 4.28 ± 0.18 nm. Applicants also show that the magnitude of this peak wavelength red-shift correlates with the concentration of nucleic acids, suggesting the feasibility of semi-quantitative detection of bacterial pathogens. Through integration of the bacterial enrichment and sensing, Applicants effectively drive down assay limit of detection from ~10⁴ CFU/mL to ~10³ CFU/mL and observe a mean signal enhancement factor of 3.67 (± 1.96 SEM). Finally, Applicants successfully demonstrate multiplexed analysis of polymicrobial samples within 30 min. This integrated diagnostic platform represents a novel approach to rapid molecular diagnostic testing. The system described herein is relevant to a range of clinical applications including bloodstream infections, skin and soft tissue infections, and bacterial respiratory infections.

### Example 1.3. Methods

### Example 1.3.1. Bacterial Strains, Culture Conditions and Sample Preparation

Staphylococcus aureus (ATCC #27660), Pseudomonas aeruginosa (ATCC 27853), and E. coli K12 were each pre-cultured overnight in 5 mL Tryptic soy broth (TSB) (Becton Dickenson, Franklin Lakes, NJ) in a 50 mL conical tube (37° C., 250 rpm shaking). Next, pre-culture was inoculated 1:1000 into 25 mL fresh TSB in a 250 mL Erlenmeyer Flask, and cultured for approximately 10 h under identical conditions (37° C., 250 rpm shaking). Cultures were centrifuged (12,100 × g, 4° C. 10 min) and the supernatant was aspirated. For storage of viable bacteria samples, bacteria were resuspended in fresh TSB and 50% glycerol (1:1), aliquoted, and stored at -20° C. until use. For preparation of bacterial lysate samples, an additional PBS wash step (resuspension, centrifugation, aspiration) was incorporated to remove any excess extracellular nucleic acids. Next, bacteria were resuspended in fresh PBS and aliquoted into 2 mL Eppendorf tubes. Immediately following, bacterial samples were lysed using a micro-tube heating block (100° C., 10 min). Bacterial lysate samples were stored at -20° C. until analysis.

### Example 1.3.2. Functionalization of Magnetic Particles

In this Example, species-specific functionalized magnetic nanoparticles (MNPs) were employed to capture S. aureus and P. aeruginosa. For S. aureus, 150 nm streptavidin-coated MNPs (SV0150, Ocean Nanotech, San Diego, CA) were functionalized with biotinylated anti-S. aureus polyclonal antibodies (PA1-73174, ThermoFisher Scientific, Waltham, MA). First, 1 mg of MNPs were washed three times with PBS. Next, suspended MNPs were combined with approximately 20 µg of IgG. The mixture was incubated at room temperature for 30 min under gentle rotation. Next, the conjugated MNPs were with PBS + 0.1 % Bovine Serum Albumin (BSA) four times. Finally, the conjugated MNPs were adjusted to a final concentration of 1 mg/mL. Functionalized MNPs were stored at 4° C. until use. For P. aeruginosa, Dynabeads™ M-270 Epoxy (ThermoFisher Scientific, Waltham, MA) were functionalized with anti-lipopolysaccharide polyclonal antibodies (LS-C71709, LSBio, Seattle, WA) in accordance with the manufacturer’s protocol. Conjugated MNPs were adjusted to a final concentration of 10 mg/mL and stored at 4° C. until use.

### Example 1.3.3. Nanosensor Assembly: Nanoparticle Deposition On-Chip

Nanoparticles were dispersed onto glass slides for testing using a microfluidic printing protocol. First, standard glass slides were functionalized for 30 minutes in 10% in (3-Aminopropyl)triethoxysilane in anhydrous ethanol before rinsing three times with ethanol. 40 nm CTAB-capped gold nanorods (A12-40-780-CTAB-DIH-1-50, Nanopartz, Loveland, CO) were diluted 10x in DI water, resulting in a final concentration of 0.005 mg/mL. Nanorods were placed into the wells of a 96-well plate and the glass slide was placed with a custom holder into a Carterra Microfluidic Printer. The gold nanorods were printed at specified locations on the glass slide for 45 minutes at a flow rate of 45 uL/minute. After printing, the gold nanorod arrays were heated at 60 C for 30 minutes. The resulting gold nanorod arrays were visualized using an Olympus IX71 optical microscope. The dispersed arrays were thoroughly rinsed with anhydrous ethanol, DI water, and then dried under air.

### Example 1.3.4. Gold Nanosensor Functionalization

Peptide nucleic acid (PNA) probes targeting species-specific DNA sequences for S. aureus, E. coli, and P. aeruginosa were purchased based upon commonly used PCR primer sequences (PNABio, Thousand Oaks, CA). A 5 mm square PDMS microwell was placed over the gold nanorod arrays on the glass substrate and a pipette used for all fluid handling. For multiplexed experiments, multiple microwells were used, each atop a single gold nanorod array. For functionalization, the gold nanorods on glass slide were incubated with 1 mg/mL dithiobis succinimidyl propionate (DSP) dissolved in dimethyl sulfoxide (DMSO) for 30 minutes. This crosslinking molecule activated the gold surface for coupling of free amines on the PNA. Then, the sensor arrays were put in contact with 1 mg/mL PNA probe dispersed in Tris-EDTA buffer (pH 7.0) for 30 minutes. Transmission spectra were collected before and after conjugation to quantify successful PNA conjugation.

### Example 1.3.5. Microchip Design and Fabrication

The integrated micro-device was designed using AutoCAD 2020. The microchannel couples a “jagged” serpentine channel to a hexagonal bacterial capture region. Serpentine-based mixers have been widely utilized for high-efficiency, low-shear mixing of biological samples, as opposed to chaotic advection. Previous work has demonstrated that additional modifications to the serpentine-channel side-wall result in further enhancement to mixing efficiency, with larger width features having a more significant effect. These findings inspired the design of the jagged serpentine model described herein.

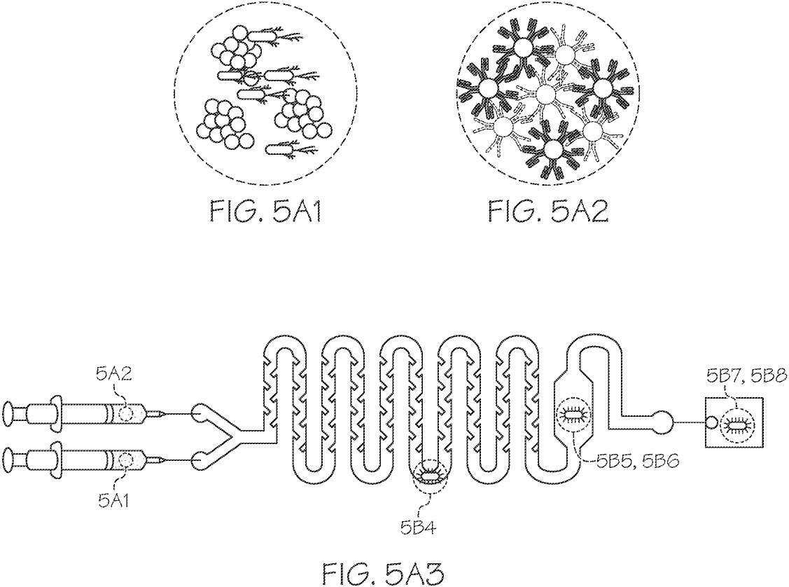

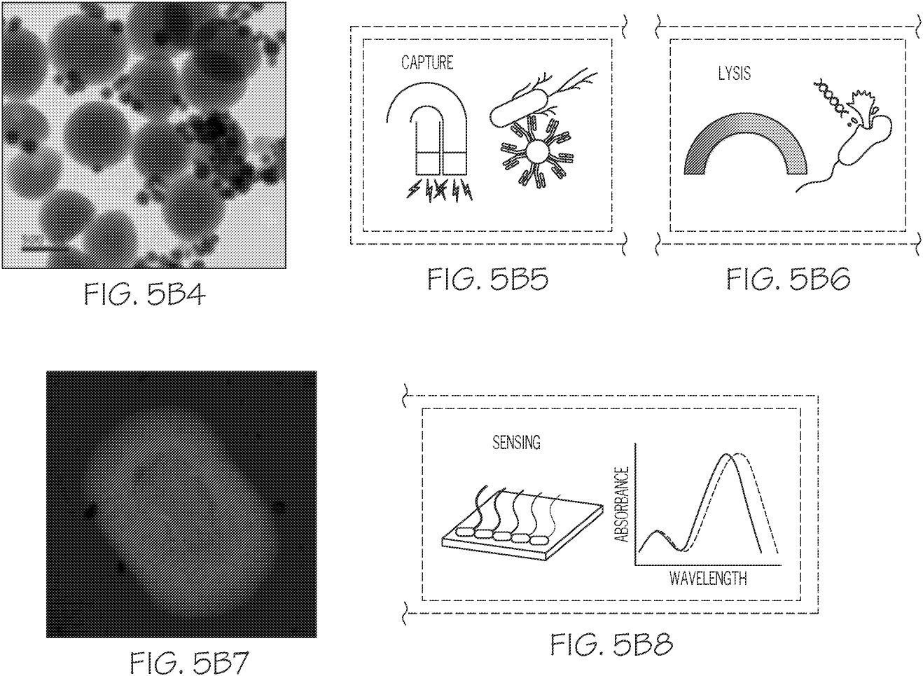

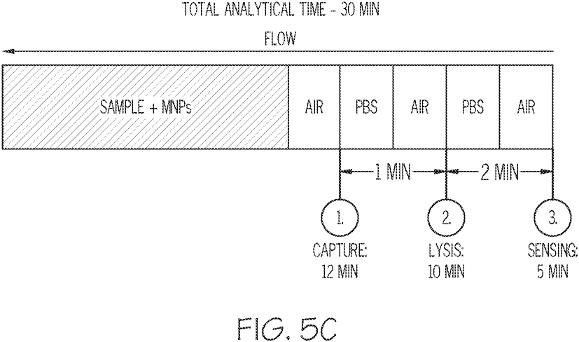

Two neodymium (NdFeB) external magnets were positioned under the hexagonal chamber (B424-N52, K&J Magnetics, Pipersville, PA). The surface area of the micro-device is approx. 14.1 cm² (70 mm × 21 mm). The serpentine channel is composed of ten turns; channel width is approximately 2 mm and channel height is approximately 100 µm. Microchannel design and dimensions are further specified in FIG. 5A1-5A3 and FIGS. B4-B8. Mixing and velocity profiles of the microchannel were characterized in COMSOL Multiphysics prior to device fabrication (FIG. 5C). A precision laser photomask was fabricated by Fine Line Imaging (Colorado Springs, CO). Next, polydimethylsiloxane (PDMS) devices were fabricated using a standard soft lithography fabrication process and bonded to 50 mm × 75 mm glass slides using oxygen plasma.

### Example 1.3.6. Sample Processing and Bacterial Quantification

Bacteria were diluted in PBS to the desired concentration and volume (1 mL). Anti-S. aureus-MNPs were diluted to a concentration 100 µg/mL and anti-Lipopolysaccharide-MNPs were diluted to a concentration of 1.5 mg/mL. Using a syringe pump (Harvard Apparatus PHD Ultra, Holliston, MA), bacteria and functionalized MNPs were pushed through the microchip in parallel at a flowrate of 100 µL/min, resulting in an effective flowrate of 200 µL/min. Next, air was pushed through the microchip at a flowrate of 200 µL/min to clear microsystem of remaining fluid, completing the bacterial capture and enrichment step. To prepare the system for bacterial lysis, 50 µL of PBS followed by air was pushed through the microchip at 100 µL/min to fill the hexagonal microchamber capture region. The external magnets were then removed and the microchip was heated via hotplate at 110° C. for 10 min, resulting in bacterial lysis. Finally, an additional 50 µL of PBS followed by air was pushed through the microsystem and collected for nanoplasmonic sensing (Section 1.4.7) (FIG. 5C).

Bacteria were quantified on TSB agar plates using traditional plate counting methods. Capture efficiency was calculated by quantifying the number of viable bacteria in the input sample and comparing it to the number of viable bacteria in the output sample. System sterilization was performed by pushing 2 mL of 70% ethanol at 100 µL/hr followed by 2 mL of PBS at 100 µL/hr. (For the integrated experimental workflow, PBS wash volume was increased to 4 mL to clear any remaining nucleic acids from the microchip). Lastly, approx. 0.5 mL of air was pushed though the microsystem to clear any remaining fluid prior to sample processing. In order to quantify potential bacterial death and/or loss within the microsystem, control samples, containing only viable bacteria (i.e., without magnetic nanoparticles), were processed on the system.

### Example 1.3.7. Gold Nanosensor Operation

The bare gold nanosensor in phosphate buffered saline (PBS) was measured before each sample. For measurement, the cell lysate sample was introduced to the microwell atop the gold nanosensor arrays. The sample was allowed to incubate with the nanosensor for 5 minutes at room temperature to allow cell nucleic acids to bind to the nanosensor before spectral collection. For multiplexed testing, the same sample was delivered atop multiple sensing arrays using a single microwell. Three spectral measurements were taken of each sample, and each spectrum contained both a signal and a background measurement together.

### Example 1.3.8. Spectral Collection and Plasmonic Peak Quantification

These spectra were collected using a FERGIE Integrated Spectrograph (Princeton Instruments) coupled to an optical microscope. A spectrum was taken with both the nanoparticle area and the background in a single measurement, so that the background could be corrected and extinction spectrum calculated. These spectra were then processed in MATLAB to calculate the extinction spectrum and peak location. The location of the peak wavelength was determined through a calculation of center of mass of peak boundaries.

### Example 1.4. Results

### Example 1.4.1. Overview of Integrated Platform

Applicants’ platform couples microfluidic immunomagnetic bacterial localization to nanoplasmonic molecular profiling, enabling characterization of bacterial samples in 30 min, eliminating the need for time-intensive culture-based steps, which require upwards of 24 hours (FIG. 5A1-5C. First, a bacterial sample is combined with magnetic nanoparticles (MNPs) that are functionalized with antibodies that target bacterial surface proteins. Bacteria and functionalized MNPs move in parallel through the microchannel. As on-chip mixing occurs, bacteria bind to functionalized MNPs. These bacteria-MNP complexes are retained in a hexagonal capture region within microchannel via an external magnet, while excess fluid exits the microchannel. Following capture, bacteria are thermally lysed, and the LSPR sensor is exposed to the concentrated bacterial lysate. If target nucleic acid sequences are present in the sample, a red shift in peak absorbance wavelength is observed (FIGS. 5A1-A3 and 5B4-B8). The total-analytical-time for the sample enrichment, lysis, and sensing workflow developed here is 30 min (FIG. C).

### Example 1.4.2. Microfluidic Immunomagnetic Bacterial Capture and Enrichment

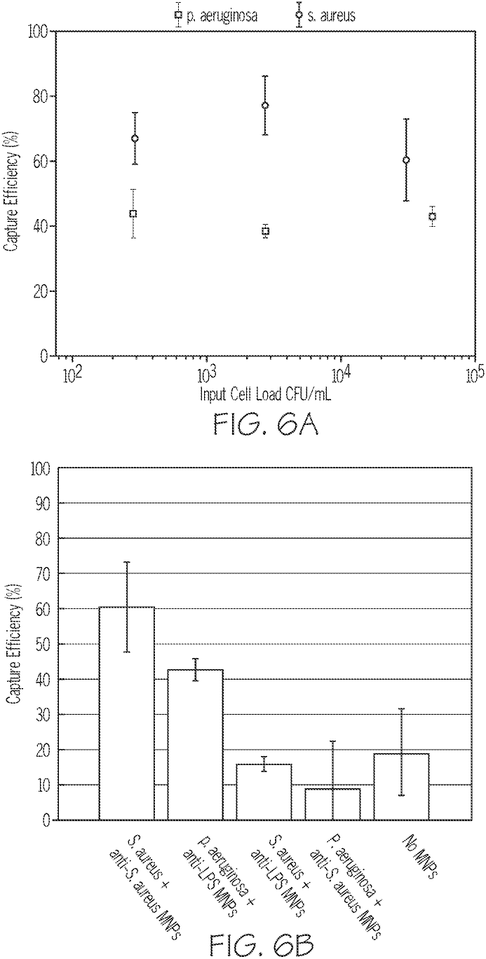

First, Applicants characterized the bacterial capture efficiency of the microsystem in two bacterial species, in addition to conducting a preliminary evaluation of capture antibody specificity. Magnetic nanoparticles were functionalized with antibodies targeting bacterial surface proteins and combined with bacterial samples. Specifically, immunomagnetic capture efficiency was evaluated for both Staphylococcus aureus and Pseudomonas aeruginosa using anti-S. aureus antibodies and anti-lipopolysaccharide antibodies, respectively. Notably, sample mixing and incubation with functionalized magnetic nanoparticles occurred on-chip in a time-window of approximately 30 seconds of residence time in the microchannel. Bacterial capture efficiency was evaluated at bacterial concentrations ranging from approximately 10² CFU/mL to 10⁴ CFU/mL (FIG. 6A). Mean bacterial capture efficiency for all reported samples was 55.0% (± 6.4% SEM). For S. aureus, capture efficiency ranged from 60.5% to 77.3% at starting bacterial concentrations of approximately 10⁴ CFU/mL and 10³ CFU/mL, respectively. For P. aeruginosa, capture efficiency ranged from 38.5% to 43.9% at starting bacterial concentrations of approximately 10³ CFU/mL and 10² CFU/mL, respectively. Although capture efficiency was significantly greater for S. aureus than P. aeruginosa, no statistically significant differences were observed in capture efficiency as a function of input bacterial concentration.

Following evaluation of bacterial capture efficiency, Applicants conducted a preliminary evaluation of capture antibody specificity to confirm limited antibody cross reactivity between the two bacterial species evaluated (FIGS. 6A-6B). Specifically, P. aeruginosa was exposed to magnetic nanoparticles functionalized with polyclonal anti-S. aureus antibodies, and S. aureus, a Gram-positive bacterium, was exposed to magnetic nanoparticles functionalized polyclonal anti-Lipopolysaccharide antibodies. Lipopolysaccharide (LPS) is a major component of the cell wall of Gram-negative bacteria. Gram-positive bacteria do not contain LPS. Statistically significant capture was not observed when compared to control samples, which contained no magnetic particles. These findings suggests limited antibody cross reactivity.

### Example 1.4.3. Species-Specific Nanoplasmonic Sensing of Bacterial Nucleic Acids

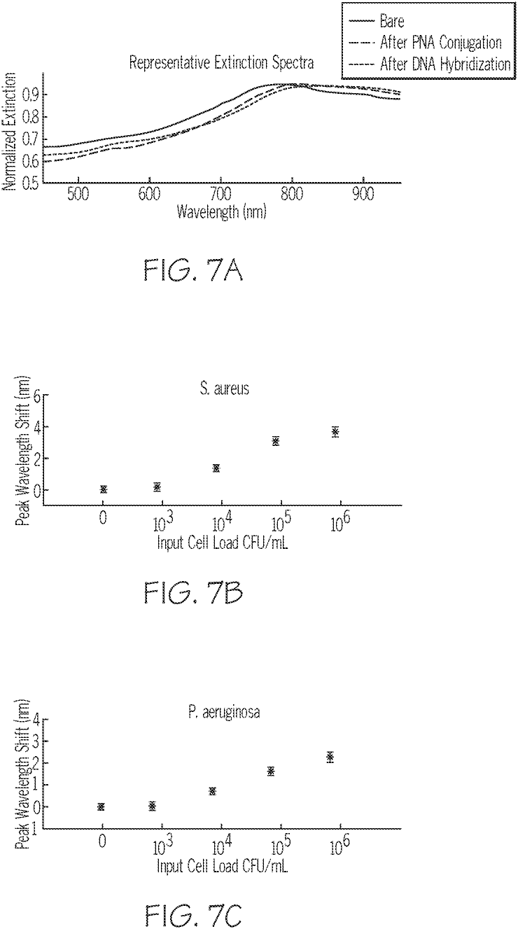

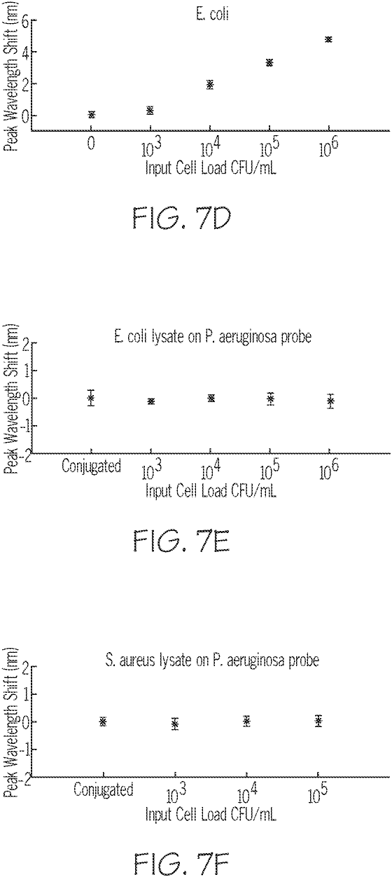

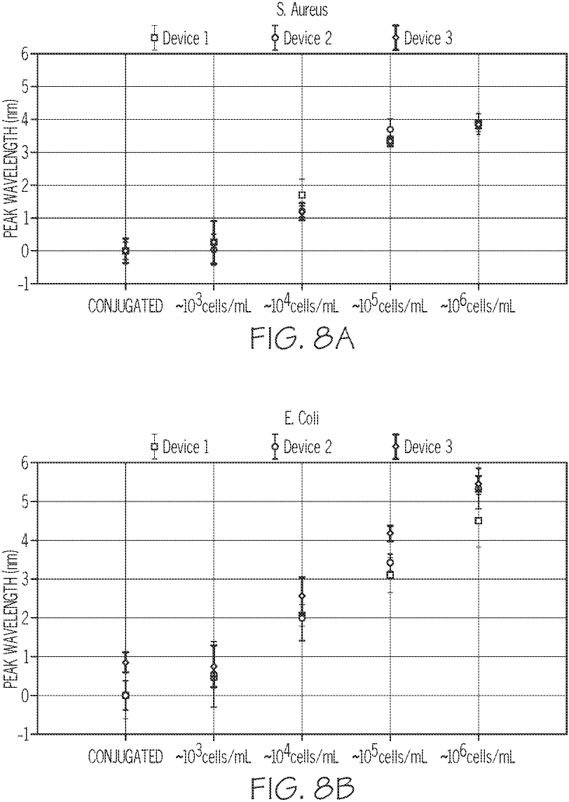

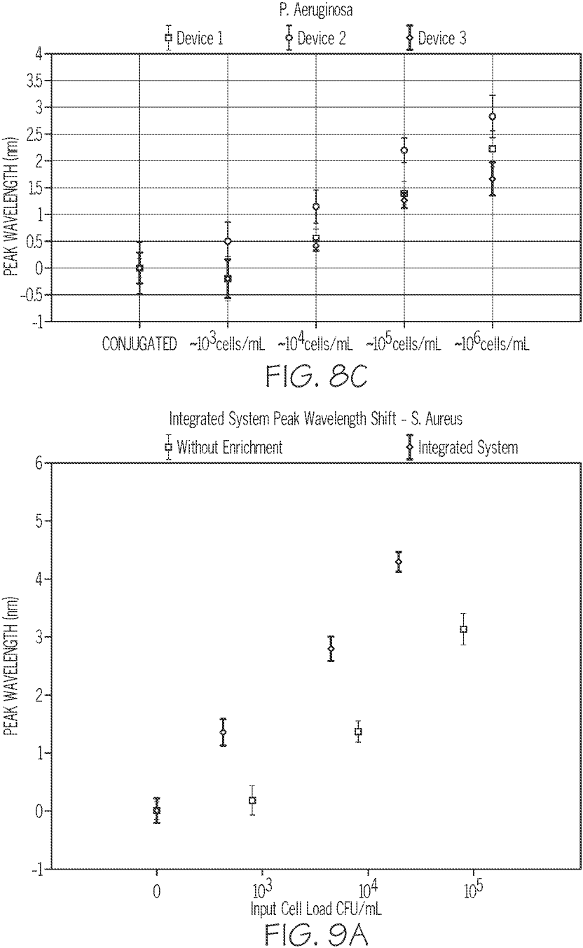

Next, Applicants demonstrated the feasibility of species-specific detection using Applicants’ nanoplasmonic biosensing platform. Colloidal gold nanorods were functionalized with species-specific peptide nucleic acid probes (PNA). Upon hybridization of a target nucleic acid sequence to a complementary PNA probe, a red-shift in peak absorbance wavelength was observed (FIG. 7A). Species-specific sensing was demonstrated in heat-lysed S. aureus, E. coli, and P. aeruginosa (FIGS. 7B-7D). In all bacterial species, a significant peak wavelength shift was first observed at a cell load of approximately 10⁴ CFU/mL. The magnitude of the peak wavelength shift successively increased with increasing bacterial concentrations, suggesting the feasibility of semi-quantitative sample characterization. Lastly, PNA probe specificity was confirmed through a series of negative control experiments in which a species-specific sensor was exposed to lysate from an off-target bacterium (i.e., P. aeruginosa sensor exposed to E. coli lysate) (FIGS. 7E-7F). In these control experiments, no significant peak wavelength shift was observed, suggesting probe specificity to target bacteria. Lastly, strong data reproducibility was observed (FIGS. 8A-8C).

### Example 1.4.4. Integration of Bacterial Enrichment and Nanoplasmonic Detection

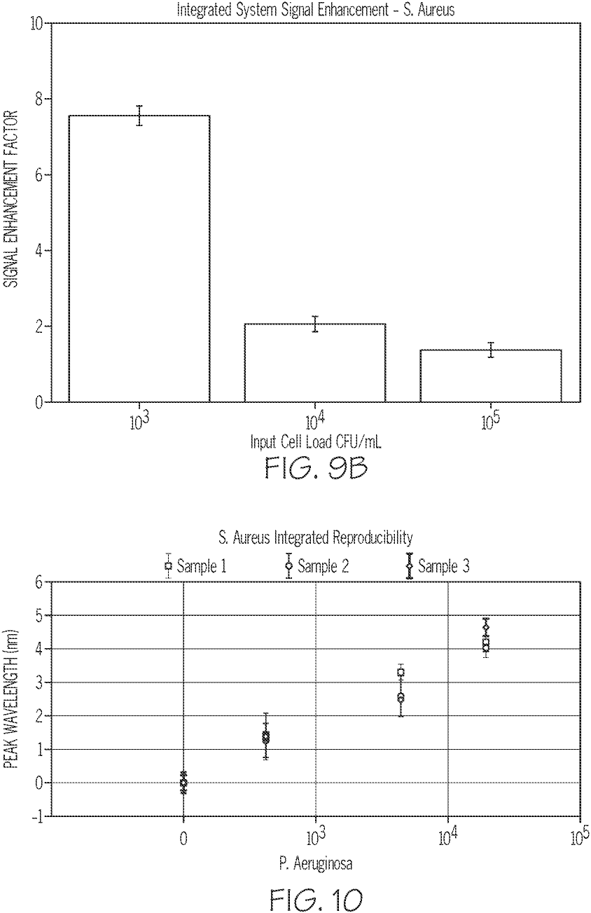

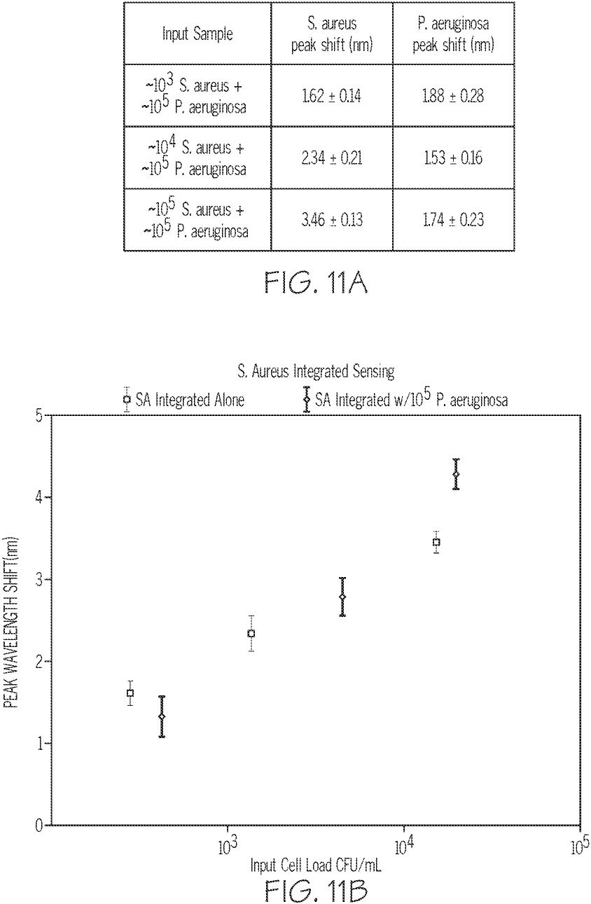

Following discrete analysis of both bacterial capture efficiency and species-specific nanoplasmonic sensing, Applicants proceeded to characterize the integrated enrichment and detection system. Through integration, Applicants observed an ~10-fold increase in platform sensitivity, effectively decreasing the limit of detection from ~10⁴ CFU/mL to ~10³ CFU/mL (FIG. 9A). Further, integration of the discrete capture and detection elements increased the magnitude of the peak wavelength shift. Applicants analyzed signal enhancement factor as a function of input bacterial concentration and observed a mean signal enhancement factor 3.67 (± 1.96 SEM) (FIG. 9B). The signal enhancement factor ranged from 1.37 to 7.58 for bacterial concentrations of approximately 10⁵ CFU/mL and 10³ CFU/mL, respectively. Lastly, strong data reproducibility on Applicants’ integrated enrichment and detection system was also observed (FIG. 10).

Next, Applicants moved to evaluate the feasibility of multiplexed characterization of polymicrobial bacterial samples. In these experiments, Applicants combined a fixed concentration of P. aeruginosa (~10⁵ CFU/mL) with varying concentrations of S. aureus (~10³, 10⁴, 10⁵ CFU/mL). Polymicrobial bacterial samples were processed in parallel with a mixture of functionalized magnetic nanoparticles, which contained both anti-S. aureus MNPs and anti-LPS MNPs. Following isolation and lysis, bacterial lysate was exposed to a LSPR sensing array; the sensing array is composed of spatially disparate, species-specific nanoplasmonic sensors. As expected, the peak wavelength shift for P. aeruginosa remained constant, while the magnitude of peak wavelength shift for S. aureus increased with increasing bacterial load (FIG. 11A). Specifically, the mean peak wavelength shift for P. aeruginosa was 1.72 nm ± 0.13 nm, and the mean peak wavelength shift for S. aureus ranged from 1.62 nm ± 0.14 nm to 3.46 nm ± 0.13 nm for bacterial concentrations of approximately 10³ CFU/mL and 10⁵ CFU/mL, respectively. Notably, analysis of polymicrobial samples had no significant observable effect on the signal intensity (i.e., magnitude of peak wavelength shift) compared to a single-species sample (FIG. 11B). This finding suggests the feasibility of semi-quantitative analysis of polymicrobial samples.

### Example 1.5 Discussion and Conclusions

To the best of Applicants’ knowledge, this is the first study to couple immunomagnetic bacterial isolation to species-specific nanoplasmonic sensing of bacterial nucleic acids. Applicants demonstrate multiplexed bacterial capture coupled to species-specific nanoplasmonic sensing. Further, Applicants validate this platform in polymicrobial samples and demonstrate the feasibility of multiplexed, semi-quantitative samples analysis. Notably, by coupling enrichment and detection steps into a single assay — effectively concentrating analytes of interest — Applicants are able to drive down the assay limit-of-detection by approximately 10-fold.

Previous work by Applicants’ group has shown high-throughput immunomagnetic isolation of both circulating tumor cells (CTCs) and S. aureus. That said, this in the first Example where sample mixing and incubation with functionalized magnetic nanoparticles occur entirely on-chip, minimizing required sample handling steps and dramatically shortening total-analytical time. Due to the high-cost and poor stability associated with antibodies, future efforts will investigate the use of aptamers for whole-cell isolation.

Prior work by Applicants’ group has demonstrated LSPR sensing of circulating tumor DNAs (ctDNA), but this is the first report of employing these functionalized gold nanoparticles for species-specific LSPR sensing of bacterial nucleic acids. Future efforts will explore optimization of nanoparticles geometry and size to increase detection sensitivity. Given the successful validation of the multiplexed platform using two the bacterial species described in this Example, future studies will also incorporate additional species-specific probes of key disease-causing microorganisms, in addition to including sequences to identify key antibiotic resistance genes.

The Example presents a microsystem that couples bacterial enrichment and localization to species-specific nanoplasmonic sensing of bacterial nucleic acids. In addition to being rapid and high-throughput, Applicants’ micro-scale platform can conduct multiplexed, semi-quantitative characterization of polymicrobial samples, which is relevant to range of clinical indications including bacterial respiratory infections, bloodstream infections, skin and soft tissue infections. Moving forward, Applicants aim to characterize platform efficacy in complex biological matrices to evaluate its feasibility for use in clinical samples.

### Example 2. Multiplexed Quantification of KRAS Circulating Tumor DNA Using Nanoplasmonic Arrays

This Example describes a multiplexed quantification of KRAS circulating tumor DNA using nanoplasmonic arrays according to aspects of the present disclosure.

In this Example, Applicants demonstrate the development of a nanoplasmonic sensor array for multiplexed capture and quantification of circulating tumor DNA without amplification. The platform is capable of sensing three mutations in exon 2 of the KRAS gene within 10 minutes of sample delivery to the microfluidic sensor. For sensor fabrication, arrayed spots of unconjugated gold nanorods were deposited using bidirectional microfluidic printing, allowing for even dispersion of the colloidal nanorods onto an activated glass slide substrate. This unique approach to nanosensor fabrication allowed for individual sensing spots for each relevant mutation, demonstrating the ability to test for a panel of mutations. The rods were subsequently functionalized with peptide nucleic acids complementary to the G12D, G12R, and G12V mutations in the KRAS gene. Mixed samples of synthetic circulating tumor DNA spiked into patient serum samples were then flowed over the sensor using a microfluidic channel and allowed to incubate for 10 minutes. A range of ctDNA concentrations were tested to determine sensitivity, with a sensor limit of detection less than 10 ng/mL. The data agreed excellently with electromagnetic simulations of conjugated and bound on-chip nanoparticles. This paper demonstrates a rapid and robust methodology for nanosensor fabrication and for quantifying multiple sequences of ctDNA on-chip directly from a patient sample without amplification. This approach can extend to detection of clinically relevant ctDNA panels on a single chip.

### Example 2.1. Materials and Methods

### Example 2.1.1. Overall Workflow

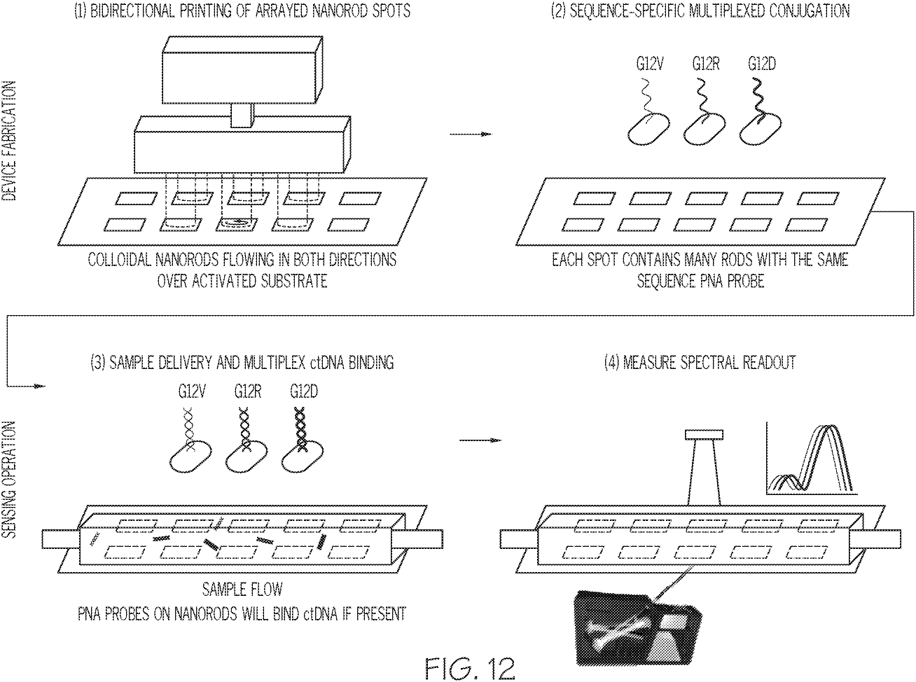

FIG. 12 shows the fabrication and operation of the plasmonic arrays for multiplexed sensing. Firstly, a microfluidic printer (Carterra continuous flow microspotter) is used to make an array of gold nanorod spots. Then, each spot is individually functionalized to capture a unique ctDNA sequence of interest. Following conjugation, a microfluidic channel is placed over the conjugated spots, and the sample is allowed to incubate with the sensor. Finally, each individual spot is measured for calculation of resonant peak shift and spectral readout. This workflow allows for fabrication of the on-chip ctDNA sensor and operation. Through this process, one can fabricate and read out the concentration of multiple sequences of ctDNA simultaneously with a single sample delivery.

### Example 2.1.2. Glass Slide Functionalization

Bare glass slides (VWR) were functionalized in 10% APTES (3-Aminopropyl triethoxysilane 99%) by volume in anhydrous ethanol. The slide and solution were incubated together for 10 minutes before the slides were rinsed three times with pure ethanol and dried. This results in a positive charge on the glass slides, promoting electrostatic interactions with the negatively charged gold nanorods for the microfluidic printing step. The resulting hydrophilicity of the slide could be verified by pipetting a drop of water onto the slide and observing surface tension changes.

### Example 2.1.3. Microfluidic Printing of Nanoparticles On-Chip

150 uL of gold nanorods (40 nm × 124 nm, resonance peak = 780 nm, Nanopartz Inc) at a concentration of OD 0.25 were pipetted into a 96 well plate. Both glass slide and the well plate were placed into the Carterra continuous flow microspotter. Before printing, a flow test was run each time to ensure that all of the tubing was working properly. A print was run in a specified location on the glass slide for 45 minutes at a flow rate of 45 uL/min. Printed spots could be easily visualized by the naked eye, or through optical or electron microscope imaging. SEM imaging was performed using a Tescan Vega3 SEM, and optical imaging was conducted on an Olympus IX71 equipped with a computer-controlled CCD digital camera (DP72).

### Example 2.1.4. On-Chip Conjugation With PNA Probes

After taking a baseline transmission, the gold nanorods were functionalized for selective ctDNA capture. For these studies, Applicants used PNA probes specific to the relevant mutations in the KRAS gene (PNABio). The PNA probes used for this Example were 5′-TAC GCC ATC AGC TCC (SEQ ID: 01; G12D), 5′-TAC GCC ACG AGC TCC (SEQ ID: 02; G12R), and 5′-TAC GCC AAC AGC TCC (SEQ ID: 03; G12V). Each of these probes was 15 base pairs long and complementary to the mutation of interest, with the mutation centered. A prior study conducted within Applicants’ group conducted thermodynamic simulations to improve selectivity to point mutations, a technique which could be employed in future work.

The conjugation steps were adapted from a protocol for coating gold foil from ThermoFisher Scientific. First, the gold nanorods on glass slide were incubated with 2.5 mg/mL DSP (dithiobis succinimidyl propionate), a cross linker, in DMSO (dimethyl sulfoxide). The DSP served as a stable cross linker onto the gold surface and provided active NHS for free amine coupling. This incubation occurred for 30 minutes before washing with DMSO and then water. Then coupling of 1 mg/mL PNA probe in Tris-EDTA buffer (10 mM Tris-HCl and 0.1 mM EDTA, ThermoFisher) was performed for 1 hour. The surface was rinsed with buffer and ready to be put into contact with synthetic ctDNA or the patient sample.

### Example 2.1.5. Device Operation and ctDNA Measurement

After the nanorod spots were functionalized, a microfluidic chip was placed on top of them and bonded to the glass slide for sample delivery. Synthetic double stranded ctDNA oligos with 41 base pair length were ordered to match the G12D, G12R, and G12V sequences with mutations centered (IDT DNA). The sequences were as follows: 5′ — ACT TGT GGT AGT TGG AGC TGA TGG CGT AGG CAA GAG TGC CT (SEQ ID: 04; G12D), 5′ — ACT TGT GGT AGT TGG AGC TCG TGG CGT AGG CAA GAG TGC CT (SEQ ID: 05; G12R), 5′ - ACT TGT GGT AGT TGG AGC TGT TGG CGT AGG CAA GAG TGC CT (SEQ ID: 06; G12V). These oligos were diluted to concentrations of 25 ng/mL, 50 ng/mL, 75 ng/mL, and 100 ng/mL spiked into health patient serum. The sensing spots were then put into contact with the different concentrations of the complementary mutated synthetic ctDNA oligos using the microfluidic channel. The sensing spots were incubated with the synthetic ctDNA solutions for 5 minutes to allow binding before spectral measurement.

### Example 2.1.6. Optical Setup and Spectrum Collection

Optical spectra were taken using a setup containing a FERGIE Integrated Spectrograph (Princeton Instruments) mounted onto an Olympus microscope. The microscope white light source was used as the spectrometer light source with all filters removed, and the spectrograph was mounted to the port. The microscope was focused so that the nanorod sensing area was centered within the frame with some bare glass slide within the frame of view. All spectra were collected through the transparent PDMS microchannel on the glass slide. Then a spectrum was collected with the spectrometer slit in place and a center wavelength of 700 nm. All intensity data were saved in raw form, and a single spectral measurement captured the spectra of both the signal area (containing the nanorods) and the background (absent the nanorods). This intensity data by pixel was exported in matrix form to MATLAB for processing.

### Example 2.1.7. Electromagnetic Simulation

To calculate the expected resonance shifts associated with gold nanorod LSPR modes, Applicants developed 2D electromagnetic simulations using Lumerical. First, Applicants studied bare gold nanorods (40×124 nm, same dimensions as used experimentally), with no surface coating. Applicants then modeled the PNA conformal layer as a conformal monolayer with a thickness of 6.5 nm and a refractive index of 1.46, and the bound PNA+DNA as a conformal monolayer with a thickness of 5.7 nm and a refractive index of 1.59. This accounts for the change undergone as the single stranded PNA shortens upon hybridization of target DNA, and represents the difference in refractive index between single- and double-stranded DNA.

### Example 2.1.8. Spectral Analysis for Resonance Peak

The data outputted from the spectrometer contains 256 pixel value rows and 1023 wavelengths (ranging from ~421 nm to ~ 985 nm) as columns. The sample (i.e., signal) and background area were selected from the CCD image and the heatmap of intensity values. The sample area contained rows where the sensor was present, and the background was the bare glass slide without nanoparticles.

A custom MATLAB script was designed for data processing. The extinction was calculated from the transmittance. These data were then used to find the resonance peak. The resonance peak was found using the wavelength corresponding to the center of mass from the bounds of the peak. The center of mass was calculated which provided the resonant peak wavelength for each of the spectra.

The data were smoothed using Lowess smoothing before plotting the curves with the shifted peaks. For each sample measurement peak wavelength output, three sensors were fabricated, and the peak wavelength shift averaged. Spectral shifts were calculated by subtracting the peak location when in contact with DNA from the peak of the bare sensor. The extinction curves and calculated peak locations were plotted with error-bars representing the standard error of the mean.

### Example 2.2. Results and Discussion

### Example 2.2.1. Multiplexed Plasmonic Sensing On-Chip

Almost all methods of ctDNA capture and analysis involve amplification to produce enough DNA material to then characterize and quantify sequences. Plasmonic sensing provides an alternative, amplification-free method of sequence-specific ctDNA sensing. This methodology relies on the standing electromagnetic waves at the surface of a metal and a dielectric that are sensitive transducers of refractive index change. Prior work describes selective capture of ctDNA sequences using gold nanorods functionalized with peptide nucleic acids (PNAs) complementary to the sequence of interest. This Example was done with nanorods in solution for one particular sequence of interest, making it hard to multiplex and test for multiple sequences at once. The extension of plasmonic sensing to multiplexed applications allows for rapid capture of a range of clinically relevant biomarkers at once. This Example employs nanorods as the sensing unit however this process can easily be extended to new geometries of plasmonic nanoparticles, potentially with higher sensitivity.

A common format for multiplexed diagnostics involves 96-well plates with a range of individual reactions and samples deposited in them. While this is effective for laboratory work, it poses fluid handling challenges that could be improved by thoughtful integration. Multiplexed plasmonic sensors are platforms with spatially separated readout “spots” that are each conjugated to target a unique biomarker, akin to microarrays. The sample can then be delivered to all the sensing spots and read out at once, allowing for minimal sample preparation and fluid handling. Advances in microfluidics and chip design have streamlined this process, allowing for operation using a much smaller sample size (µL) and efficient ctDNA capture, enrichment, and quantification step.

### Example 2.2.2. Microfluidic Printing and Spectra

For integration of plasmonic sensing onto a single chip, both nanolithography and patterning of colloidal particles have been previously explored. Nanolithography allows for fine control of nanoscale features but can be expensive and resource intensive. There are a number of methods of patterning colloidal nanoparticles on-chip, including spin coating, dip coating, and even simple pipetting combined with evaporation. When particles are to be dispersed on chip, simply pipetting them and allowing the solvent to evaporate often results in a phenomenon called the coffee-ring effect, in which the particles disperse to the edges of the droplet rather than being evenly dispersed in the pipetted spot. This clustering of particles along the edge of the spot hinders the application of these spots to plasmonic sensing, which works best with well-defined plasmonic spots, having well-separated nanorods which reduce undesirable particle near-field coupling and resonance broadening effects. A promising alternative to these methods which avoids the coffee ring effect is microfluidic printing of nanoparticles onto substrates. Bidirectional microfluidic printing removes the evaporation effect associated with patterning and allows particles to evenly fill defined spaces.

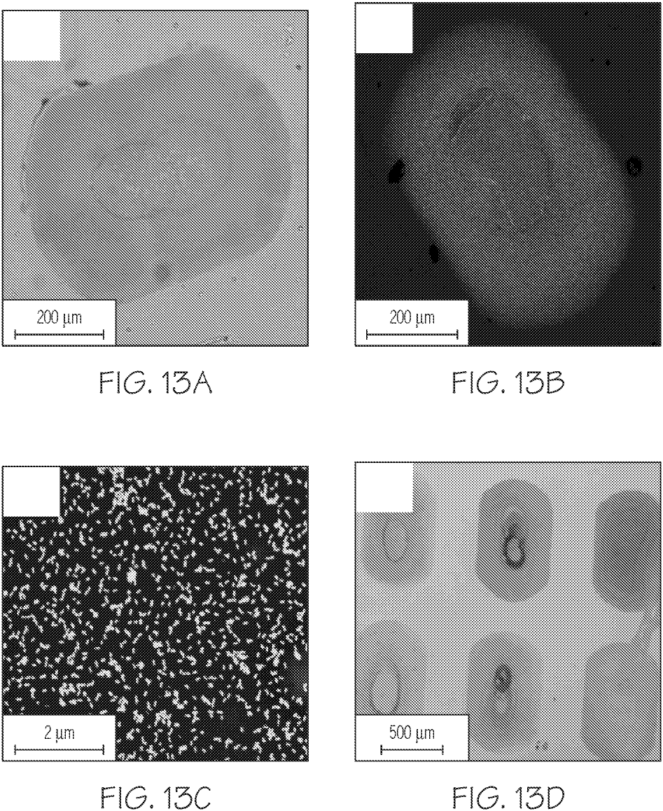

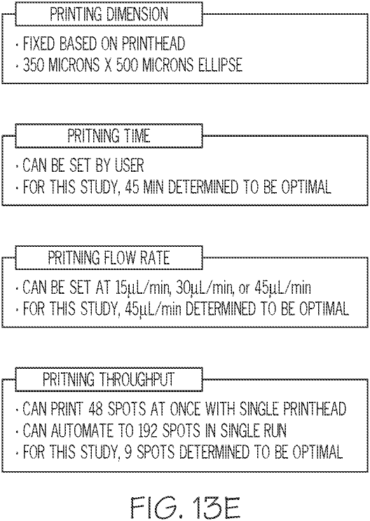

An initial fabrication test was conducted by simply pipetting rods onto a glass slide and allowing them to evaporate, which resulted in the rods moving to the edge of the spot, as expected by the coffee ring effect. Microfluidic printing methodology was developed to allow for even dispersion of nanorods within the set spot area without any clustering of the rods around the edge. Examples of the printed spots fabricated using the Carterra Continuous Flow Microspotter can be seen in FIG. 13. The printed spot geometry is defined by the microspotter specifications and prints uniform spots that are 350 by 500 microns. Insets FIG. 13A and FIG. 13B each show an individual spot using optical and SEM imaging. A crisp boundary and uniform color can be observed, indicating that the rods are uniformly dispersed through the entire spot. Inset FIG. 13C shows the dispersion of rods zoomed in, and it can be observed that the nanorods are randomly dispersed with consistent spacing between them. All of these spots could also be observed by eye after printing, allowing rapid troubleshooting and microchannel alignment atop the fabricated array.

The printing process was optimized to avoid challenges such as ineffective deposition due to lack of glass slide surface functionalization and inconsistent dispersion of gold nanorods in solution within the 96-well plate. The combination of glass slide functionalization with a positive charge and testing a range of concentrations of nanorods helped to overcome these challenges. The glass slide functionalization allowed for favorable electrostatic interactions between the negatively charged nanorods and the positively charged glass slide. In determining the ideal concentration of nanorods to use for spectral sensing, a range of nanorod concentrations from optical density (OD) 0.25 to OD 25 were evaluated. The optimal concentration for resonance spectra figure of merit was determined to be OD 0.25, which had the largest amplitude extinction peaks, indicating minimized near-field coupling. Once this printing process was optimized, and array of spots was printed, as shown in inset FIG. 13D, allowing for multiplexed capture with a separate PNA probe on each of the spots. The developed microfluidic printing process outlines a method to pattern a glass slide with hundreds of sensing spots, each functionalized for a different analyte of interest (FIG. 13E).

### Example 2.2.3. Surface Conjugation

Once the bare gold nanorods were patterned on the chip, a functionalization process was conducted to attach peptide nucleic acid probes to each of the nanorod spots. Nanoparticle conjugation is often conducted in solution, with the nanoparticles dispersed in a liquid and mixed in a tube. While this is a valid option for solution-based tests, integration with microfluidics allows for spatial multiplexing and enhanced mixing between the patient sample and the functionalized nanoparticles. To this end, the patterned nanorods were functionalized in microwells after they were printed into various spots on the glass slide.

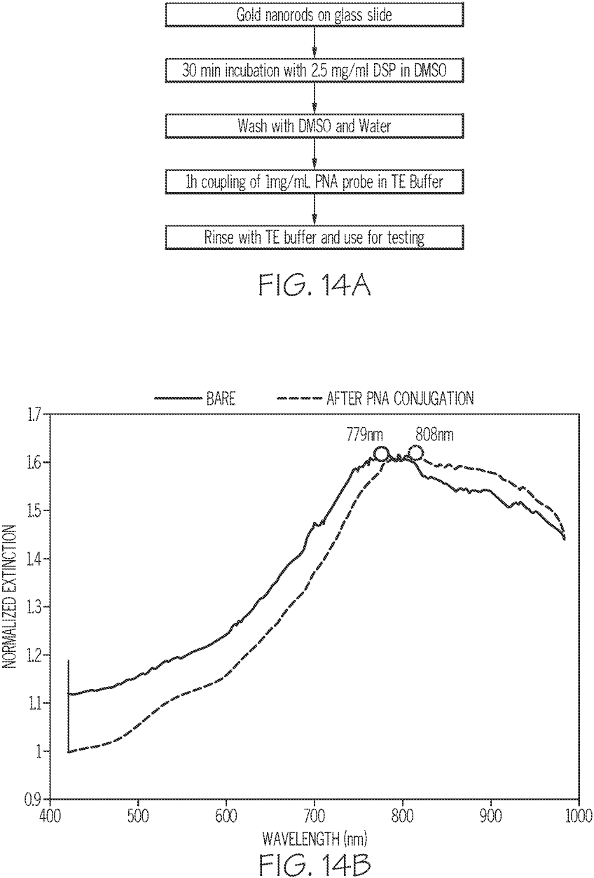

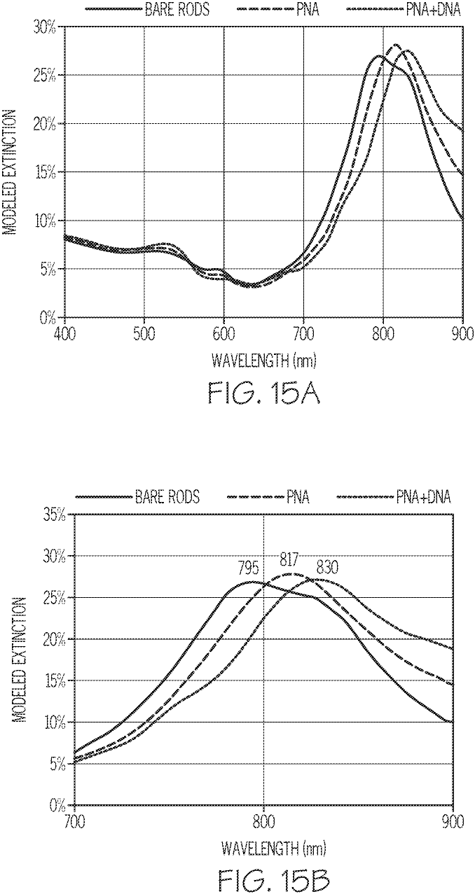

The first step of the conjugation involves activating the gold to bind with free amines on the PNA, before incubation with the PNA itself and rinsing with buffer before testing. This workflow can be seen in flowchart form in FIG. 14A. In order to verify whether the conjugation was successful, the conjugation was performed on the spectrometer setup. For each workflow, a spectrum was taken of the bare gold nanorods on the slide, then before the incubation with DSP in DMSO, and then after the conjugation. As additional layers coupled to the nanorods, successive redshifts could be observed in the resonance peak, due to increased loading of the plasmonic nanorod antenna. An example of the extinction spectrum of on-chip patterned gold nanorods before and after PNA conjugation can be seen in FIG. 14B. A clear shift in the resonance peak from 779 nm before functionalization to 808 nm after functionalization can be observed.

### Example 2.2.4. Two Dimensional Electromagnetic Simulation

Applicants’ electromagnetic simulation sought to capture the effects of successive conformal layers atop the on-chip gold nanorods after conjugation and binding to ctDNA. Applicants took into account both the length and refractive index of the PNA layer and the bound PNA-ctDNA hybrid. This allows us to anticipate the expected LSPR shift after both conjugation with a PNA and then subsequent binding of ctDNA. The geometry of the simulation was configured to represent dispersed nanorods on a substrate, similar to Applicants′ on-chip arrays. A 2D array of nanorods with spacing in the x and y directions of X nm was used in order to avoid both near-field coupling and far-field diffractive effects, thus approximating a single isolated particle. Furthermore, it has previously been shown that well-dispersed random nanoparticles exhibit single-particle behavior, validating Applicants’ modeling approach.

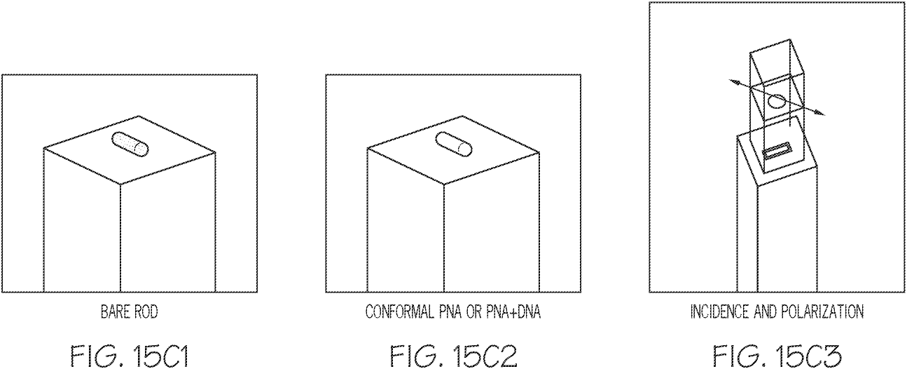

This small simulation study provided excellent qualitative backing for Applicants’ experimental findings. From the simulation results, Applicants determined about a 20 nm peak shift after conjugation (FIG. 15B) which agrees qualitatively with Applicants’ experimentally determined value (FIG. 14B). Applicants then saw a subsequent sensing working range of about 10 nm until the sensor was fully bound. This gives us an idea of the maximum peak shift Applicants can expect to see upon binding to ctDNA in solution.

### Example 2.2.5. Multiplexed ctDNA Sensing

Once the multiplexed nanorod spots were conjugated, they were put in contact with the samples of synthetic ctDNA diluted to known concentrations. When in contact, the analytes of interest were incubated with the rods and if present, bound to the PNA probes on the surface of the rods. The incubation times were tested by taking spectra every minute for 30 minutes and plotting the maximum shift at a high concentration of synthetic oligos. From this Example, it was shown that a five minute incubation time in the microfluidic channel was enough to see the full shift in spectral peak. Once this was determined, the sensor was put into contact with serum samples with a range of synthetic ctDNA concentrations from 0 to 100 ng/mL in increments of 25 ng/mL. These are relevant to the clinically relevant range of ctDNA circulating in patient blood at a later stage of gastrointestinal cancers.

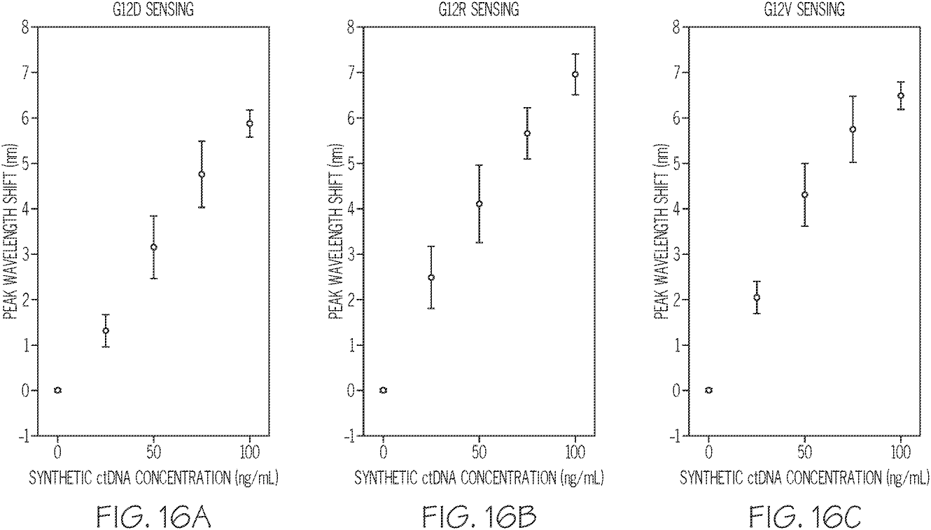

After putting the sensor in contact with solutions of a range of synthetic ctDNA concentrations, the spectra were collected and peak locations calculated. The synthetic ctDNA was spiked into healthy patient serum samples, to examine potential for nonspecific binding as well as interference from a clinical sample. For FIG. 16, only one sequence of interest was spiked into the serum in each test. As expected, a linear relationship between the concentration of ctDNA and the peak location was seen (FIG. 16). This data was collected for each sequence of interest on 3 nanorod spots on 3 chips and compiled with error bars that represent the standard error of the mean. The trend of linear relationship between synthetic ctDNA concentration and sensor output holds across sequences as well, as shown in insets FIG. 16A, FIG. 16B, and FIG. 16C, the linear working range of the sensor approached that of the clinically relevant ctDNA concentration range, making this sensor and its fabrication process a promising methodology for potential clinical use.

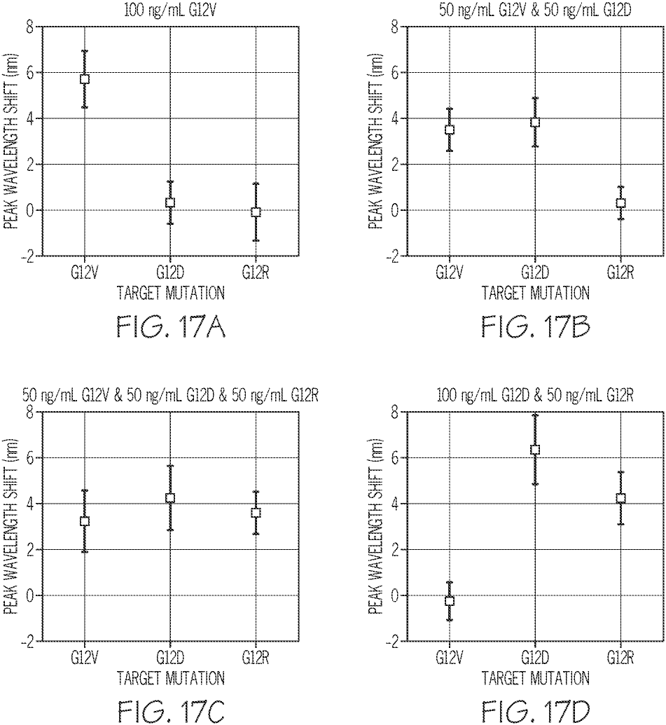

FIGS. 17A-D illustrate multiplexed sensing of 3 mutations in the KRAS gene. Peak wavelength shift is calculated as the difference between peak wavelength before and after ctDNA addition. Each data point represents measurements on three sensing spots conjugated and put in contact with relevant targets. Error bars represent standard error of the mean. FIG. 17A shows sensing measurement of all three conjugated spots, with only G12V synthetic DNA present. FIG. 17B shows mixed sample of G12V and G12D variant showing no binding to G12R sensor. FIG. 17C shows mixed samples of all three variants showing approximately equal binding. FIG. 17D shows mixed samples of G12D and G12R synthetic DNA showing semi-quantitative discrimination between wavelength output.

Applicants also conducted an additional study with mixed samples of synthetic ctDNA in buffer. Applicants showed extremely minimal nonspecific binding (i.e. the G12V sequence did not bind to the G12R array spot) and the ability to be semi-quantitative about relative mutational loads. Through this study Applicants also demonstrated that Applicants would see peak shifts for multiple of the sequences if multiple were present. The exact sequences shown here could never exist at the same time, given that they are in the same gene location, but these data show the promise of this technique for detection of multiple clinically relevant sequences at once. These data show that if there is a mixed population of ctDNAs, as can be expected in human cancers due to tumor heterogeneity, this sensor would be able to quantify the concentrations of each sequence of the population. It also shows the ability to discriminate point mutations within this system. Because the resolution of this spectrometer is a fraction of a nanometer, this means that the limit of detection of this sensor is in the range of a several ng/mL, and that it is able to discriminate between concentrations in this range. This Example can be extended for capture of multiple unrelated mutation sequences, and for discrimination of single base pair changes from the wild type sequence.

### Example 2.3. Conclusions

Applicants demonstrated a novel methodology for the development of a multiplexed, on-chip plasmonic array for liquid biopsy that included a method of microfluidic printing of nanorod spots onto a glass slide and a conjugation methodology for sequence-specific detection of ctDNA for liquid biopsy. First, bidirectional microfluidic printing was performed for even dispersal and concentration control of gold nanorods onto a functionalized glass slide. This allowed for high throughput printing of evenly dispersed plasmonic spots which overcame common barriers in nanoparticle dispersion including the coffee-ring effect. Individual spots of gold nanorods were then chemically functionalized with PNA probes for sequence-specific capture of clinically relevant mutations within the KRAS gene. The sensor was put into contact with serum samples spiked with known concentrations of synthetic ctDNA, and the extinction spectrum through the sample was measured. For all three sequences tested, a linear relationship between synthetic ctDNA concentration and resonant peak location was found, with a limit of detection approaching the clinically relevant range. This Example demonstrates a simple methodology for fabrication and operation of a multiplexed on-chip plasmonic sensor for liquid biopsy. This technology lays the groundwork for amplification-free ctDNA panel characterization and quantification from patient plasma and serum samples.

### Example 3. Plasmonic Sensing by Probe Shortening

This Example describes plasmonic sensing by probe shortening according to aspects of the present disclosure.

### Example 3.1. Overview

Applicants’ novel nanosensor concept — plasmonic molecular ruler-based sensing —couples nanoplasmonic sensing to a simplified colorimetric readout via dark field imaging. This concept relies on measurable coupling effects between plasmonic nanoparticles and a gold nanofilm upon binding of target nucleic acid sequences. Rationally designed plasmonic nanoparticles are tethered to a gold nanofilm by peptide nucleic acid probes complementary to the target RNA/DNA. Following binding of target RNA/DNA, the probe shortens into a double helix, and the proximity increases between the gold nanoparticles and gold plasmonic substrate. The coupling of colloidal particles to the gold substrate results in enhanced resonance sensitivity, which can be read out as a color change on a simple dark field image, or as a spectral change in the transmittance/reflectance/extinction/absorbance.

Applicants’ preliminary work demonstrates on-chip sample preparation, optimization of PNA-nanoparticle chemistry, and successful optical detection of target nucleic acid sequences, all of which are utilized. The proposed Example involves the completion of two technical tasks towards the development of this novel nanosensor. First, Applicants aim to optimize nanoparticle geometries and PNA probe sequences to maximize target RNA/DNA capture and plasmonic coupling enhancement. Next, Applicants will couple Applicants’ nanoplasmonic sensing platform to a simplified dark field readout system to demonstrate the successful detection of sequence-specific RNA at clinically relevant concentrations.

### Example 3.2. Technology

The innovation in the proposed Example focuses on the development of a novel nanoplasmonic detection platform. More specifically, Applicants’ novel nanosensor concept —plasmonic molecular ruler-based sensing — couples nanoplasmonic sensing to a simplified colorimetric readout via dark field imaging. This concept relies on measurable coupling effects between plasmonic nanoparticles and a gold nanofilm upon binding of target nucleic acid sequences, and allows for the sensitive and specific detection of target viral RNA sequences. Localized surface plasmons on metal (e.g. gold) nanoparticles are extremely sensitive to small changes at their surface, and can be employed to enhance surface sensitivity for a variety of measurements. Plasmonic sensing has been demonstrated to be sensitive to a single molecule binding to a single nanoparticle. These surface plasmons exhibit enhanced resonance intensity when near other plasmonic surfaces, and particle coupling can be used to measure the presence of target biomarkers through biorecognition. This amplified intensity enables extremely sensitive detection of rare analytes (e.g. nucleic acids). Dark field microscopy allows for visualization of these light absorbance changes caused by binding events through an image capture. By using nanoparticles of finely controlled dimension, geometry, and chemistry, Applicants can detect molecular binding events through a simple dark field image.

### Example 3.3. Product

Applicants’ product is a portable device that takes a patient sample and identifies the presence of target RNA/DNA. Patient samples (either directly or in buffer) are processed on Applicants’ microfluidic chip to immunomagnetically isolate viral particles. Following capture and lysis, RNA/DNA hybridizes to plasmonic nanoparticles functionalized with peptide nucleic acid probes, and presence is read out via dark field imaging or spectral measurement. From start to finish, the product aims to limit total-analytical-time to less than 20 minutes, allowing for a rapid, point-of-care diagnosis.

### Example 3.4. Technical Overview

Applicants’ platform takes a patient sample, isolates and localizes viral particles over Applicants’ nanosensor, lyses the viral capsid, and performs species-specific RNA detection. This exact workflow could be used with lysed bacterial or mammalian cells or cell free DNA. The device readout hardware will be designed to be compact, and enable a rapid, sample-to-answer workflow from a disposable microchip. Current diagnostic methods rely on the time-consuming PCR processes to amplify target nucleic acids. Applicants’ technology eliminates the need for nucleic acid amplification through an ultrasensitive RNA/DNA detection modality, providing an answer within minutes.

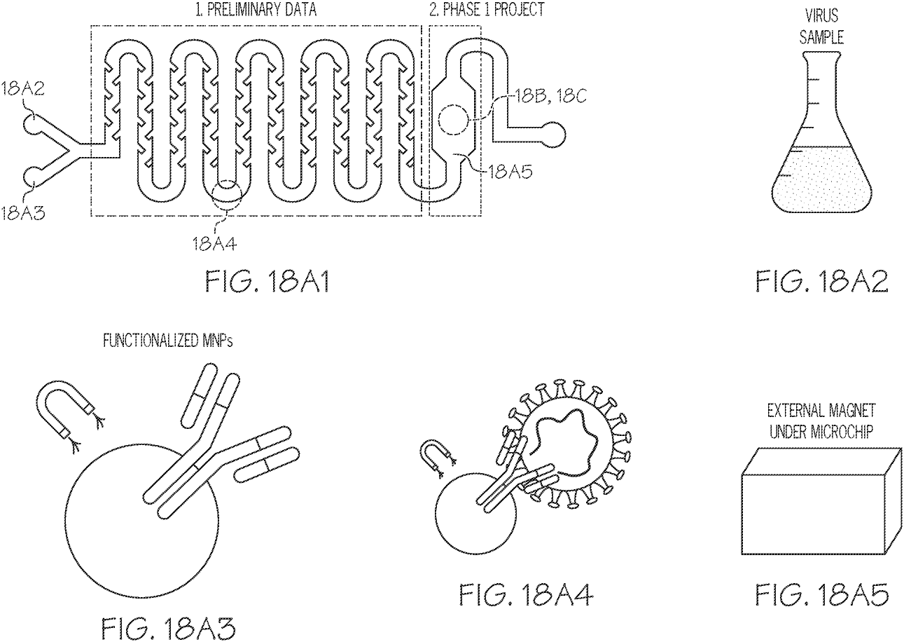

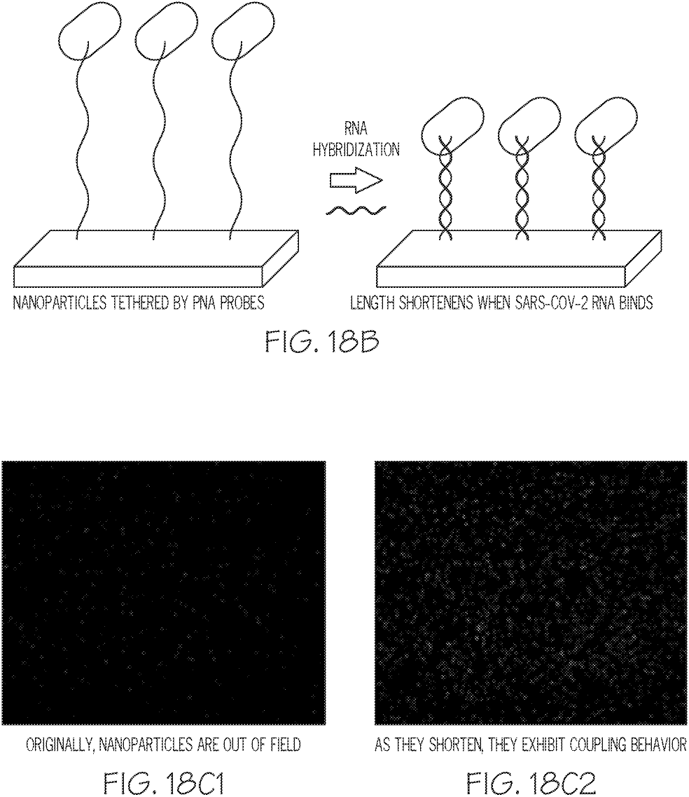

FIG. 18A1-18C2 illustrate an overview of a proposed detection mechanism. FIG. 18A1-18A5 show a microchip design showing Phase I focus on the capture and transduction of RNA binding. FIG. 18B shows that initially nanoparticles are tethered to the gold film by PNA probes. If SARS-CoV-2 RNA is present, binding will occur, and shorten the length of the tether. FIG. 18C1-C2 show that if PNAs are unbound, the longer tether remains out of the plasmonic electric field decay length, but if PNAs bind to target RNA, the tether shortens, plasmonic coupling occurs, and binding can be visualized on dark field image.

### Example. 3.5 Theoretical Platform Development and Analysis

A finite difference time domain simulation was developed using CST Microwave Studio to investigate electric field enhancement as a function of nanoparticle geometry. The simulation will consist of a 200 nm gold film, a spacer layer (which represents the length of the PNA), and a gold nanoparticle atop the spacer. Applicants simulated nanocubes, nanorods, and nanospheres to determine the quality and coupling factors. At a base case using a nanocube, this configuration shows a much higher quality factor than simple functionalized nanoparticles, as is indicated by the narrow and high amplitude resonance peak. By changing the spacer layer thickness, Applicants can simulate the resonance shifts in the reflectance spectrum, which will translate to shifts in color in the dark field image.

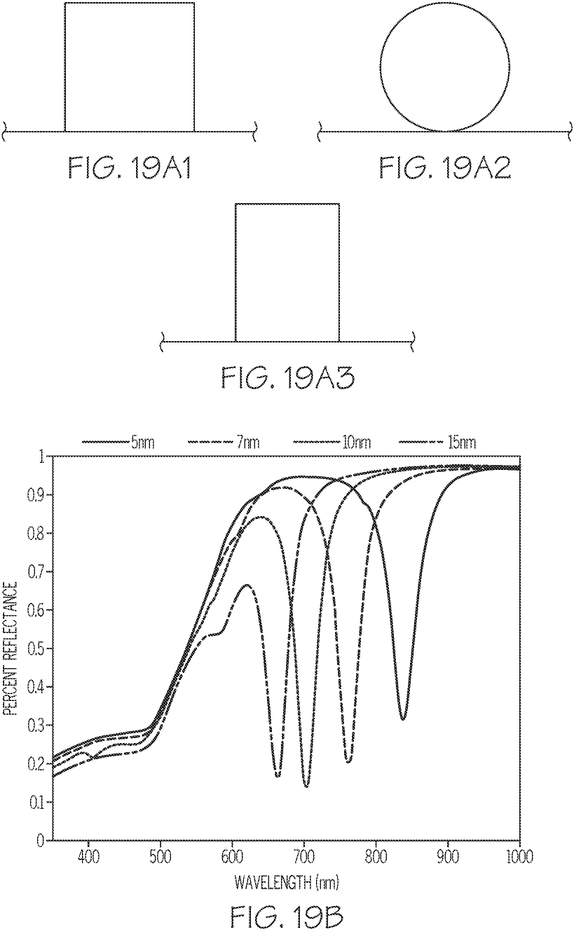

FIG. 19A1-18B illustrate a nanoparticle-on-film simulation overview. FIG. 19A1-A3 show three geometries of nanoparticles to be tested: nanocube, nanosphere, and nanorod. FIG. 19B shows preliminary CST simulation data, showing extremely high quality resonances with large peak shifts (hundreds of nm) from small (2-10 nm) thickness changes.

### Example 4. Microfluidic Enrichment of Bacteria Coupled to Contact-Free Lysis On a Magnetic Polymer Surface for Downstream Molecular Detection

This Example describes a microfluidic enrichment of bacteria coupled to contact-free lysis on a magnetic polymer surface for downstream molecular detection according to aspects of the present disclosure.

Applicants report on a microsystem that couples high-throughput bacterial immunomagnetic capture to contact-free cell lysis using an alternating current magnetic field (AMF) to enable downstream molecular characterization of bacterial nucleic acids. Traditional methods for cell lysis rely on either dilutive chemical methods, expensive biological reagents, or imprecise physical methods. Applicants present a microchip with a magnetic polymer substrate (Mag-Polymer microchip), which enables highly controlled, on-chip heating of biological targets following exposure to an AMF. First, Applicants present a theoretical framework for the quantitation of power generation for single-domain magnetic nanoparticles embedded in a polymer matrix. Next, Applicants demonstrate successful bacterial DNA recovery by coupling (1) high-throughput, sensitive microfluidic immunomagnetic capture of bacteria to (2) on-chip, contact-free bacterial lysis using an AMF. The bacterial capture efficiency exceeded 76% at 50 ml/h at cell loads as low as ~10 CFU/ml, and intact DNA was successfully recovered at starting bacterial concentrations as low as ~1000 CFU/ml. Using the presented methodology, cell lysis becomes non-dilutive, temperature is precisely controlled, and potential contamination risks are eliminated. This workflow and substrate modification could be easily integrated in a range of micro-scale diagnostic systems for infectious disease.

### Example 4.1. Introduction

Microfluidic platforms have emerged as a popular alternative to traditional macro-scale diagnostic methods. Microfluidic systems enable extremely precise fluid control and manipulation and have demonstrated their ability to isolate and detect rare cells from both environmental and biological samples by harnessing a variety of physical and chemical separation methods. The ability to rapidly isolate and specifically detect bacterial pathogens has applications in infectious disease, biosecurity, and food and water quality monitoring. Integrated micro-scale systems could aid in shortening diagnostic timelines due their demonstrated efficacy as high-throughput, sensitive, and specific biomarker isolation and detection platforms.

Numerous pathogen characterization methods rely on access to intracellular proteins and nucleic acids, requiring cell lysis following pathogen isolation. Traditional methods for cell lysis rely on either dilutive chemical methods (e.g., detergents), expensive biological reagents (e.g., lysozyme), or imprecise physical methods. There is a significant need for a rapid, precise, and reagent-free bacterial lysis method that can be easily integrated with upstream microfluidic enrichment processes. This need for highly controlled, non-dilutive cell lysis becomes especially relevant when targeting the isolation and analysis of rare cells, which is relevant to a range of clinical scenarios including the diagnosis of bloodstream infections and prosthetic joint infections.

Here, Applicants utilize microfluidic immunomagnetic separation methods to rapidly and specifically capture and concentrate bacteria of interest on the surface of Applicants’ microchip. The microchip substrate is composed of a unique three-layer magnetic polymer (Mag-Polymer), which consists of single-domain magnetic nanoparticles mixed into a polydimethylsiloxane (PDMS) matrix. Mechanisms of heat generation from magnetic nanoparticles have been comprehensively studied. Most often, these studies are performed within the context cancer therapy. Specifically, these studies investigate the use of in vivo localized magnetic nanoparticles coupled to an external alternating magnetic field for the hyperthermia of cancerous cells and/or tissues. Significant effort has been dedicated to optimizing therapeutic effects through rational design of magnetic nanoparticle characteristics such as size, geometry, and composition in an effort to limit field intensity requirements. For cancer cell hyperthermia, target temperatures range from approximately 40 to 45° C.; however, in this work, Applicants aim to reach significantly higher temperatures (i.e., 80-110° C.) to enable the highly controlled, on-chip, thermal lysis of bacteria.

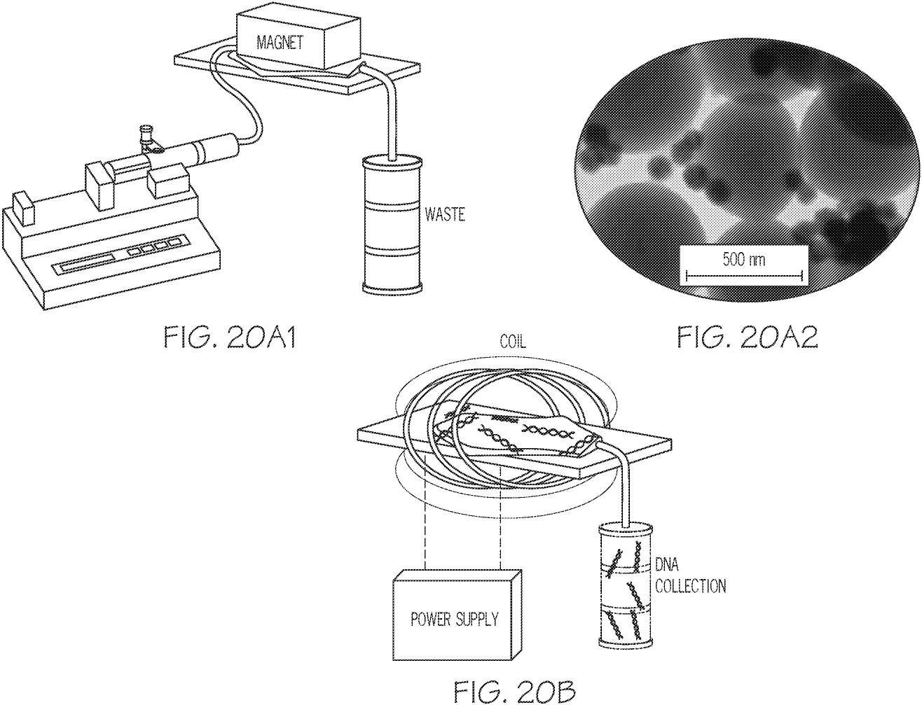

The presented methodology couples microfluidic bacterial enrichment with contact-free lysis using an AC magnetic field (AMF). Following exposure to an AMF, bacteria are thermally lysed, enabling additional on-chip and/or downstream nucleic acid amplification and analysis (FIG. 20A1-20B). First, Applicants present a theoretical framework for the optimization of Mag-Polymer microchip heating as a function of magnetic field strength, field frequency, and magnetic nanoparticle characteristics. Next, Applicants demonstrate an optimized microfluidic bacterial immunomagnetic enrichment system, which enables high-throughput sample processing, while achieving extremely low limits-of-detection. Finally, Applicants provide an experimental characterization of microchip heating and demonstrate successful recovery of double-stranded bacterial DNA for downstream molecular characterization.

### Example 4.2 Bacterial Strains and Cultureconditions

Staphylococcus aureus (ATCC #27660) was pre-cultured overnight in 5 ml tryptic soy broth (TSB) (37° C., 250 rpm shaking) (Becton Dickenson, Franklin Lakes, NJ). The pre-culture was inoculated 1:1000 into 25 ml fresh TSB in a 250 ml Erlenmeyer flask and cultured for 12 h under identical conditions (37° C., 250 rpm shaking). The sample was centrifuged (12 100×g, 4° C. 10 min), and the supernatant was aspirated. Bacteria were resuspended in fresh TSB and 50% glycerol (1:1), aliquoted, and stored at -20° C. until use.

### Example 4.3. Functionalization of Magnetic Nanoparticles

150 nm streptavidin coated magnetic nanoparticles (SV0150, Ocean Nanotech, San Diego, CA) were functionalized with biotinylated anti-S. aureus polyclonal antibody (PA1-73174, ThermoFisher Scientific, Waltham, MA). First, magnetic nanoparticles (MNPs) were washed three times with PBS. Next, approximately 20 µg of IgG was added to 1 mg of suspended MNPs. The mixture was incubated for 30 min at room temperature with gentle rotation. Finally, conjugated MNPs were washed four times with 0.1% bovine serum albumin (BSA) in PBS and adjusted to a final concentration of 1 mg/ml. Functionalized MNPs were stored at 4° C. until use.

### Example 4.4. Mag-Polymer Microchip Fabrication

To fabricate the magnetic polymer, 30 nm iron oxide (Fe₃O₄) nanoparticles (Nanostructured & Amorphous Materials Inc., Katy, TX) were mixed with Sylgard-184 polydimethylsiloxane (PDMS) (Dow Corning, Midland, MI) to create a 35% (w/w) mixture. The curing agent was added to the mixture at a ratio of 1:5 (w/w). The mixture was manually stirred and degassed for 60 min. The mixture was then spin coated onto a glass slide at 600 rpm for 30 s and baked at 150° C. for 10 min. This step was repeated a total of three times to create three polymer layers, having a total thickness equal to ~200 µm. The three-layer polymer structure was selected after experimentation with varying layering and weight density structures.

### Example 4.5. Sample Preparation, Processing, and Quantification

To prepare a sample, S. aureus was diluted in PBS to the desired concentration and volume (1 ml) and combined with functionalized MNPs. Samples were incubated for 1 h at room temperature with gentle rotation. Samples were pushed through the microchip at flow rates ranging from 5 ml/h to 50 ml/h with a syringe pump (Harvard Apparatus PHD Ultra, Holliston, MA). Flow rate optimization experiments were conducted at a bacterial load on the order of 10³ CFU/ml and were combined with 25 µg functionalized MNPs per sample. Magnetic nanoparticle mass optimization experiments were conducted at bacterial load on the order of 10³ CFU/ml at a flow rate of 10 ml/h. System sensitivity experiments were conducted at a flow rate of 50 ml/h and were combined with 100 µg functionalized MNPs per sample. Bacteria were quantified using traditional plate counting methods on TSB agar plates. Capture efficiency was calculated by comparing the number of viable bacteria in the input sample to the number of viable bacteria in the output sample. Paired control samples, containing viable bacteria and without magnetic nanoparticles, were processed on the system to quantify potential bacterial loss and/or death within the microsystem. System sterilization was performed by pushing 5 ml of 70% ethanol at 0.5 ml/h, followed by 10 ml of PBS at 1 ml/h and ~2 ml of air to clear the microsystem prior to sample processing.

### Example 4.6. AC Magnetic Field, DNA Quantification, and Cell Viability

The AMF induction coil used in these experiments was a single-turn solenoid coil which was custom built by the Hoopes lab at Dartmouth College. It is powered by a 25-kW generator (Radyne, Milwaukee, WI) and cooled by a 3-ton ethylene glycol cooling system (Tek-Temp Instruments, Croydon, PA). The field was tuned to 165 kHz. The microchip surface temperature was measured using a thermal camera (Model SC325, FLIR Systems, Wilsonville, OR). Following a 60 s exposure to the AMF (200 Oe, 30 s; 500 Oe, 30 s), DNA was quantified using a Qubit 3.0 Fluorometer dsDNA High Sensitivity Assay Kit (ThermoFisher Scientific, Waltham, MA). Cell viability was determined using the plate counting method via 10 µl drop plates.

### Example 4.7. Theoretical Framework for Magnetic Polymer Heating

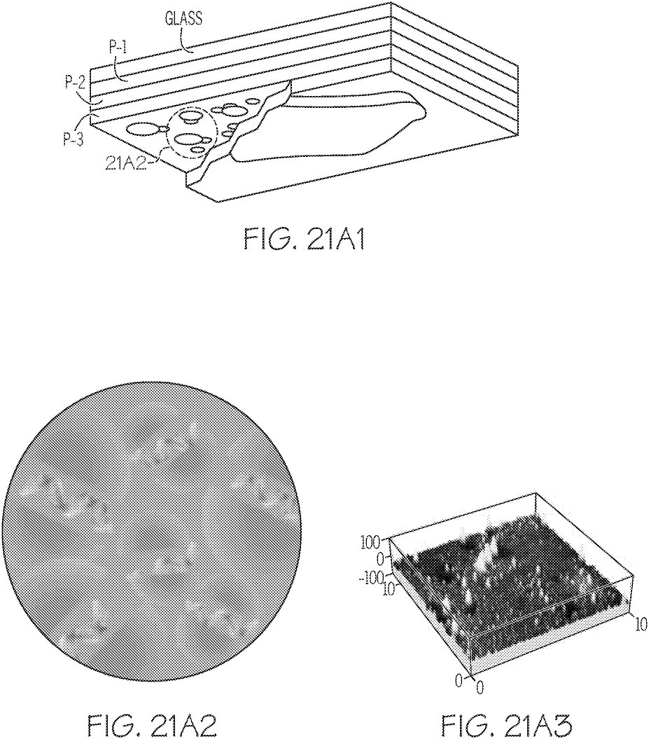

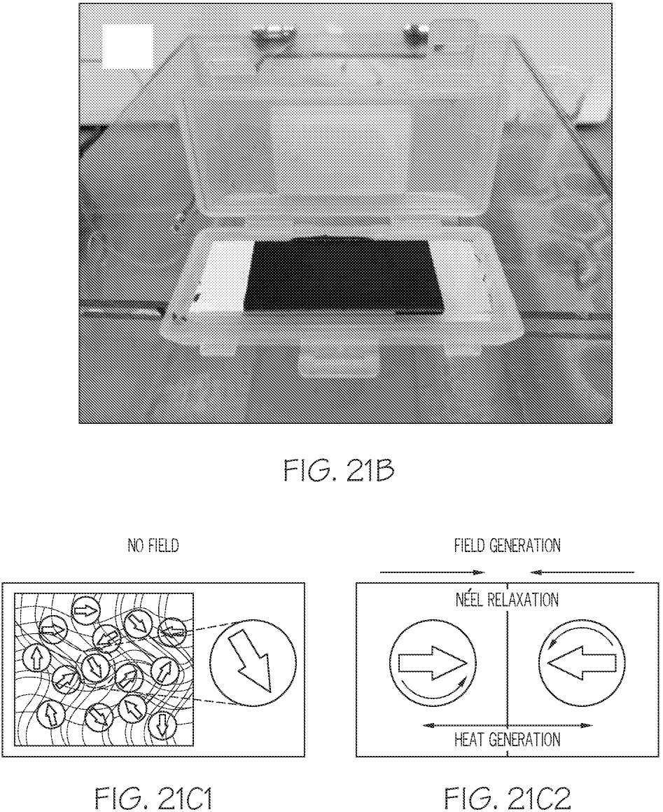

Previous work by Tong et al. identified that magnetic iron oxide particles ranging from 30 to 40 nm have a specific absorption rate (SAR) approaching the theoretical limit when exposed to a clinically relevant alternating magnetic field (AMF). Thus, ~30 nm iron oxide nanoparticles were selected as the magnetic component of Applicants’ polymeric material. These magnetic nanoparticles were spiked into polydimethylsiloxane (PDMS); this magnetic polymer was spin-coated onto a glass substrate to create a three-layer polymer structure for microchip heating (FIG. 21A1-A3 and FIG. 21B). The design of the heating layers was guided by an attempt to maximize heating efficiency, while limiting multi-layer microfabrication requirements. The three-layer polymer structure was selected as the optimal outcome. First, Applicants sought to maximize the weight density of the iron oxide in the polymer matrix, while still achieving reliable and repeatable polymer cross-linking. Next, Applicants sequentially added polymer layers until target temperatures were achieved. Importantly, Applicants wanted to preserve ease-of-fabrication and repeatability by employing a spin-coating fabrication methodology.

Below, Applicants present a theoretical framework for the quantitation of power generation from single-domain magnetic nanoparticles confined in a polymer matrix when exposed to an AMF.

Power dissipation (P) from magnetic nanoparticles following exposure to an AC magnetic field can be modeled using the Rosensweig equation,

$$P = π {μ}_{0} {x}_{0} {H}^{2} f \frac{2 π f τ}{1 + {\left(2 π f τ\right)}^{{2}^{′}}} \qquad \text{(Equation 1)}$$

where µ₀ is the permeability constant of free space (4π × 10⁻⁷ N/A²), χ₀ is the magnetic susceptibility of the particles, H is the magnetic field strength, f is the magnetic field frequency, and τ is the effective relaxation time. When exposed to an alternating magnetic field, magnetic nanoparticles produce heat via three main mechanisms: hysteresis, Brownian motion, and Néel relaxation. Given the paramagnetic properties of the single-domain iron oxide nanoparticles (<30 nm) and their confinement in a polymer matrix, Applicants can assume that magnetization reversal and heat generation is primarily limited to Néel relaxation (spin relaxation), which is dictated by the anisotropy energy of the nanoparticles (FIG. 21C1-C2). Therefore, τ can be defined as

$$τ = {τ}_{N} = {τ}_{0} e x p \left(\frac{K V}{{k}_{B} T}\right) , \qquad \text{(Equation 2)}$$

where τ₀ is the attempt time/period; KV, the anisotropy energy, is the product of the magnetocrystalline anisotropy (K) and particle volume (V); k_{B}T, the thermal energy, is the product of Boltzmann constant (k_{B}) and absolute temperature (T). By combining Equation (1) and Equation (2), Applicants can define power generation from a paramagnetic nanoparticle embedded in a polymer as follows:

$$P = π {μ}_{0} {x}_{0} {H}^{2} f \frac{2 π f {τ}_{0} exp \left(\frac{K V}{{k}_{B} T}\right)}{1 + {\left(2 π f {τ}_{0} exp \left(\frac{K V}{{k}_{B} T}\right)\right)}^{2 ′}} \qquad \text{(Equation 3)}$$

Aside from thoughtful and intentional particle selection, power dissipation from the magnetic polymer can be increased by increasing the strength of the magnetic field (H), and by optimizing the field frequency (f) such that f is equal to τ⁻¹.

### Example 4.8. Microfluidic Immunomagnetic Bacterial Enrichment

Bacterial samples were processed through a hexagonal-shaped microchannel (30 × 20 mm²) exposed to an optimized external magnetic field. The specifications of this platform have been previously reported for the isolation of circulating tumor cells (CTCs). In this work, the microchip glass substrate was modified with a three-layer, spin-coated magnetic polymer to enable contact-free heating of the microchip immediately following cell capture.

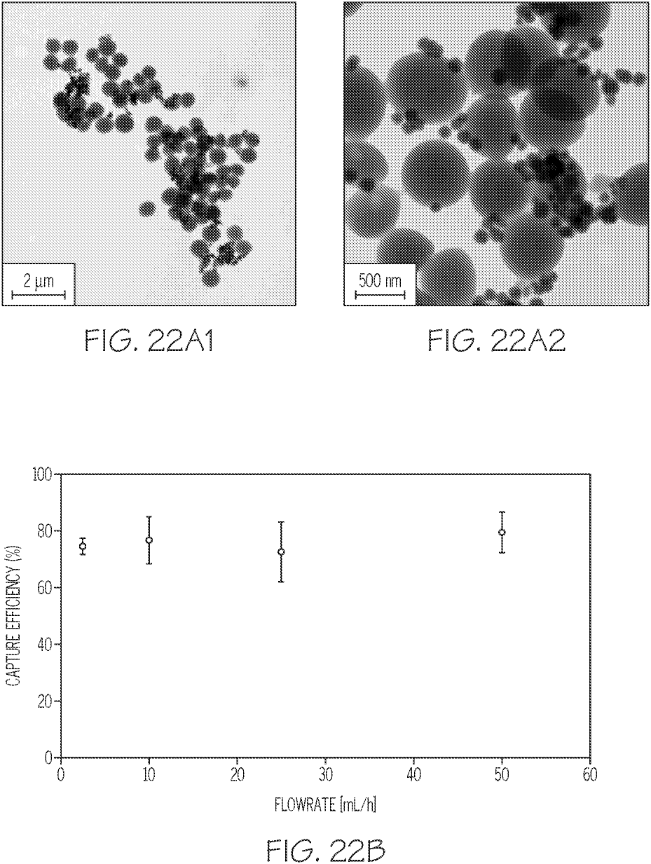

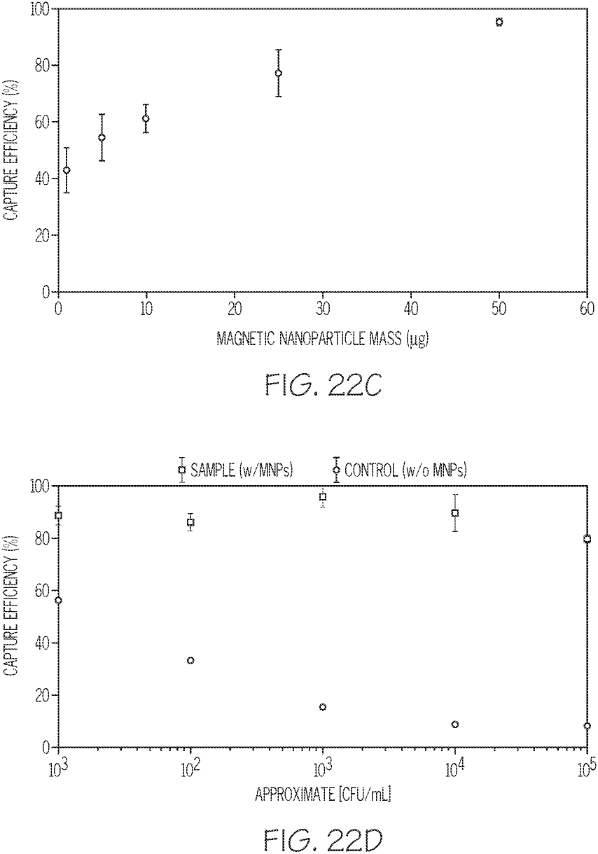

Magnetic nanoparticles for cell capture were functionalized with an anti-S. aureus polyclonal antibody to selectively bind target bacteria (FIG. 22A1-A2). The microfluidic bacterial capture system was optimized to maximize system sensitivity and sample throughput. First, bacterial capture efficiency was evaluated as a function of sample flow rate. Bacterial samples were continuously flowed through the microchannel at flow rates ranging from 5 ml/h to 50 ml/h, but no significant difference in capture efficiency was observed (FIG. 22B). This finding suggests that Applicants’ microfluidic immunomagnetic capture system is robust to high flow rates, enabling rapid sample processing and target biomarker enrichment. Next, bacterial capture efficiency was evaluated as a function of magnetic nanoparticle mass. Applicants observed that bacterial capture efficiency significantly increased with increasing magnetic nanoparticle mass (FIG. 22C). These initial experiments allowed for the implementation of optimized assay parameters to asses system limit-of-detection.

Applicants’ optimized flow-through immunomagnetic capture platform demonstrated successful bacterial capture at high flow rates (50 ml/h), while still achieving low limits-of-detection (~10 CFU/ml). By employing optimized assay conditions, Staphylococcus aureus capture efficiency ranged from 86.1% ± 3.34% to 95.93% ± 4.07% for 10⁵ CFU/ml and 10³ CFU/ml, respectively. Notably, at bacterial concentrations on the order or 10¹ CFU/ml, capture efficiency exceeded 80% for all samples evaluated, with a mean of 88.7% ± 3.49% (FIG. 22D). The data presented suggest that Applicants’ proposed immunomagnetic enrichment platform can rapidly concentrate bacteria at extremely low cell loads, which is relevant to a range of infectious disease diagnostic applications.

### Example 4.9. Microchip Heating and Quantification of Recovered DNA

Following S. aureus capture, the microchip was exposed to an AMF for 60 s (FIG. 23A). The field strength was optimized to result in a microchip temperature that maximized bacterial lysis, while preserving biological molecules of interest (i.e., dsDNA). For the first 30 s, the microchip was exposed to a field of approximately 500 Oe to rapidly achieve the target temperature (105.5° C. ± 0.92° C.). Once the target temperature was achieved, the field strength was lowered to approximately 200 Oe for an additional 30 s to maintain an exposure temperature ranging from 105.5° C. ± 0.92° C. to 100.6° C. ± 0.92° C. (FIG. 23B). In addition to the heating profile evaluated here, the magnetic polymer substrate enables extremely precise heating at a range of biologically relevant temperature profiles. In comparison to other off-chip thermal lysis methodologies (e.g., heating block), the Mag-Polymer substrate modification allows for fine-tuning of the thermal gradient and localized heating on rationally patterned regions of the microchip surface. Additionally, thermal exposure is highly homogenous and extremely precise, as is indicated by the relatively small standard error observed in reported temperatures across multiple devices. This heating modality also moves toward the design of a fully integrated microsystem for nucleic acid recovery from biological samples.

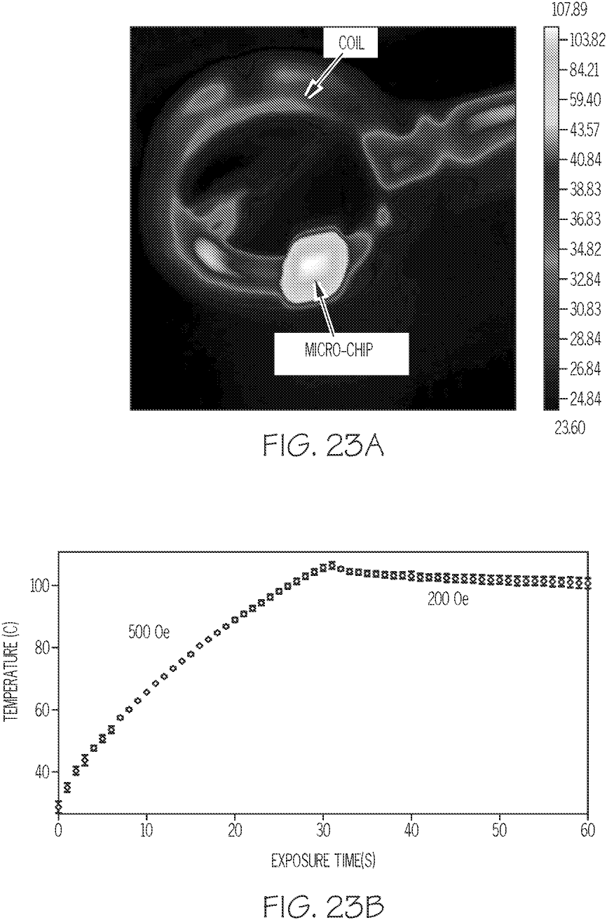

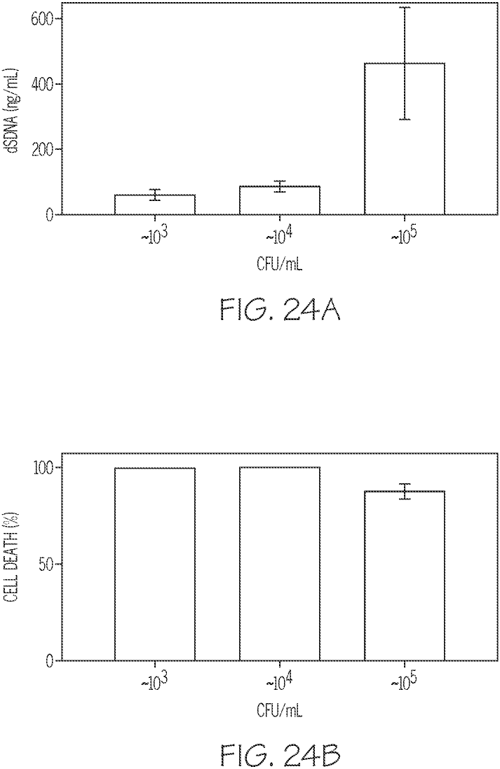

Following exposure to the AMF, the efficacy of Applicants’ contact-free cell lysis platform was evaluated as a function of recovered dsDNA and cell death (FIGS. 24A-B). Applicants demonstrate successful recovery of intact dsDNA for starting bacterial sample concentrations on the order of 10³ CFU/ml (59.8 ng/ml ± 15.2 ng/ml). Applicants hypothesize that these low detection limits are feasible as a direct result of Applicants’ microfluidic enrichment step prior to cell lysis, which effectively localizes and concentrates bacterial nucleic acids. Specifically, the starting sample volume of 1 ml is effectively concentrated to an ~5 µl sample on the surface of the microchip. Additionally, cell death was confirmed and ranged from 87.41% ± 3.95% to 99.98% ± 0.003% for bacterial sample concentrations on the order of 10⁵ CFU/ml to 10⁴ CFU/ml, respectively.

### Example 4.10. Conclusions

To the best of Applicants’ knowledge, this is the first study to report on a contact-free lysis method coupled to a flow-through microfluidic cell capture platform. Applicants describe a unique Mag-Polymer microchip design that enables controlled, contact-free, and non-dilutive cell lysis following exposure to an AMF. Applicants also provide a theoretical framework for future work aimed at optimizing power dissipation from the material in an effort to limit required external power and equipment. Applicants demonstrate extremely sensitive and high-throughput microfluidic immunomagnetic bacterial enrichment using a hexagonal microchannel and optimized external magnetic field. This enrichment platform has been previously demonstrated for the enrichment of circulating tumor cells (CTCs), but this study reports its first successful translation and application to bacterial enrichment. It is important to note that by performing bacterial enrichment prior to lysis, rare biomarker detection sensitivity is significantly increased.

Following experimental characterization of microchip heating, Applicants demonstrate that this novel methodology is successful at lysing captured bacteria and recovering intact double-stranded DNA for downstream characterization of the captured pathogen. Applicants think that this methodology is especially relevant to the micro-scale platforms that target the isolation and detection of rare biomarkers due to the ability to finely tune thermal exposure, and eliminate dilutive wash steps and/or chemical buffers. In addition to bacterial cell lysis, there are numerous other biological applications (i.e., PCR) that could utilize this Mag-Polymer microchip substrate modification and precise heating mechanism for on-chip molecular analysis and detection. Due to the relative simplicity of Applicants’ magnetic polymer substrate fabrication method, Applicants anticipate that this microchip substrate modification could be easily integrated in a range of micro-scale diagnostic systems for rapid, precise, and contact-free heating to enable comprehensive characterization of the disease-causing pathogens.

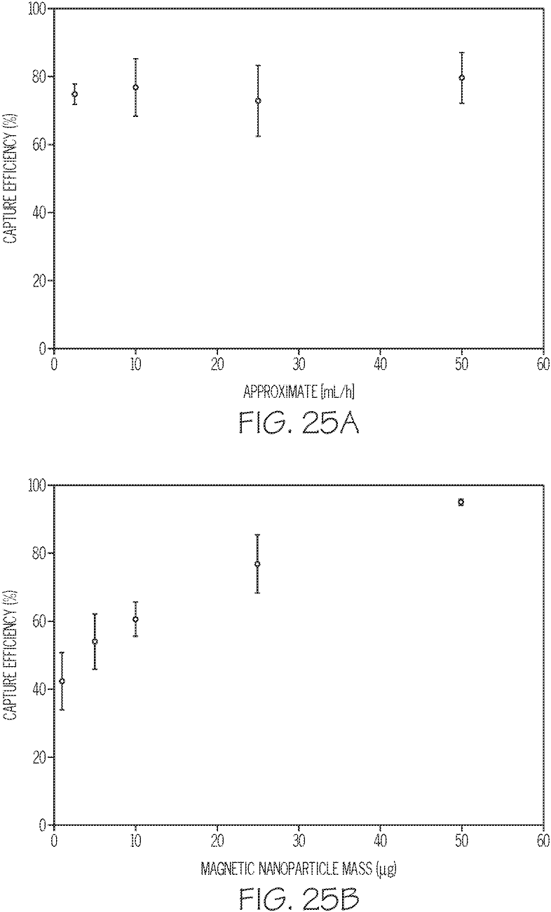

FIGS. 25A-B illustrate bacterial capture efficiency optimization. FIG. 25A shows bacterial capture efficiency as a function of flow rate. Using Applicants’ microfluidic chip, relatively high flow rates could be achieved, while preserving capture efficiency. Flowrate experiments were conducted at bacterial load on the order of 10³ CFU/mL, and with 25 µg functionalized magnetic nanoparticles. Experiments were performed in triplicate, and standard error of the mean is reported. FIG. 25B shows bacterial capture efficiency as a function of magnetic nanoparticle (MNP) mass. Increased MNP mass resulted in significantly greater bacterial capture efficiency. MNP mass optimization experiments were conducted at bacterial load on the order of 10³ CFU/mL, and at a flowrate of 10 mL/h. Experiments were performed in triplicate, and standard error of the mean is reported.

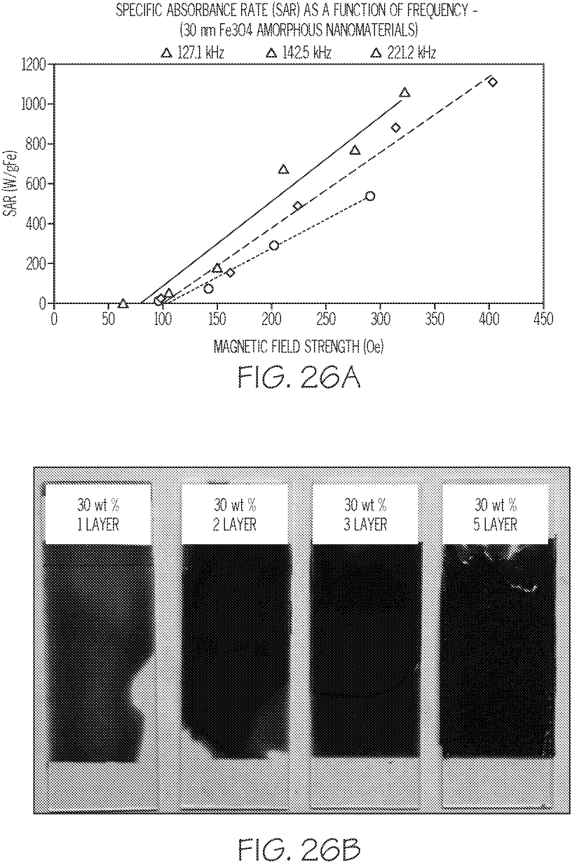

FIG. 26 illustrates magnetic polymer characterization and optimization. FIG. 26A shows characterization of specific absorbance rate of the iron oxide heating particles as a function of field frequency. SAR was characterized in water. FIG. 26B shows examples of various multi-layer magnetic polymer substrates (left to right: 1-layer, 2-layer, 3-layer, 5- layer).

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1-239. (canceled)

240. A platform comprising:
an inlet region for receiving a sample;
a mixing region for mixing the sample;
a capturing region comprising a first surface for capturing one or more components of the sample, wherein the first surface is downstream the mixing region; and
a plasmonic sensing region comprising a second surface for detecting an analyte from the sample, wherein the second surface comprises an analyte detecting agent.

241. The platform of claim 240, wherein the first surface comprises one of the following configured to immobilize vesicle capture particles:
(a) a magnetized region or a region exposed to a magnetic field;
(b) a functionalized region, wherein the functionalized region is functionalized with at least one functional group; and
(c) a porous region for size-based separation.

242. The platform of claim 240, wherein the analyte detecting agent is selected from the group consisting of aptamers, oligonucleotides, single-stranded oligonucleotides, double-stranded oligonucleotides, DNA, RNA, single stranded DNA, antibodies, peptide nucleic acids (PNAs), selective polymers, and combinations thereof.

243. The platform of claim 240, wherein the second surface comprises:
plasmonic nanostructures associated with a dielectric surface, wherein the plasmonic nanostructures are coupled to the analyte detecting agent.

244. The platform of claim 240, wherein the second surface is in a form of an array, wherein the array comprises a plurality of different analyte detecting agents that are specific for detecting different analytes.

245. The platform of claim 240, wherein the second surface is the same as the first surface.

246. The platform of claim 240, wherein the second surface is adjacent or proximal to the first surface.

247. The platform of claim 240, wherein the second surface is downstream from the first surface.

248. A method of detecting an analyte from a sample comprising:
(a) providing a platform of claim 240;
(b) flowing the sample through the platform;
(c) detecting a change in property of the second surface of the sensor; and
(d) correlating the change in property to a characteristic of the analyte, thereby detecting the analyte.

249. The method of claim 248, wherein the platform further comprises a first surface for immobilizing vesicle capture particle-vesicle complexes formed by binding vesicles in the sample with vesicle capture particles.

250. The method of claim 249, wherein the first surface comprises one of the following:
(a) a magnetized region or a region exposed to a magnetic field, wherein the region is utilized to immobilize the vesicle capture particles;
(b) a functionalized region, wherein the functionalized region is functionalized with at least one functional group, and wherein the at least one functional group is utilized to immobilize the vesicle capture particles; and
(c) a porous region, wherein the porous region is utilized to immobilize the vesicle capture particles through size-based separation.

251. The method of claim 249, wherein the first surface is the same as the second surface, which comprises plasmonic nanostructures associated with a dielectric surface.

252. The method of claim 249, wherein the second surface is downstream from the first surface.

253. The method of claim 249, wherein flowing the sample comprises flowing the sample with the vesicle capture particle.

254. The method of claim 249, wherein the sample is pre-incubated with the vesicle capture particles to form vesicle capture particle-vesicle complexes prior to the step (b).

255. The method of claim 249, wherein the first surface comprises immobilized vesicle capture particles.

256. The method of claim 249, further comprising immobilizing the vesicle capture particle-vesicle complexes on the first surface after the step (b).

257. The method of claim 256, wherein the immobilizing occurs by a method selected from the group consisting of magnet-based immobilization, pelleting, centrifugation, size-based separations, filtration, inertial separations, acoustofluidic separations, material property based separations, dielectrophoretic separations, immunoaffinity-based separation, and combinations thereof.

258. The method of claim 249, further comprising lysing the vesicles of the vesicle capture particle-vesicle complexes thereby releasing the analyte after the step (b).

259. The method of claim 258, wherein the lysing comprises applying heat to the platform, exposing the platform to an alternating magnetic field, applying a lysis material to the platform, applying a chemical lysis agent to the platform, freezing, mechanical perturbation, or combinations thereof.

260. The method of claim 258, further comprising exposing the analyte to the sensor and associating the analyte with the analyte detecting agent on the plasmonic nanostructures associated with a dielectric surface after the lysing step.

261. The method of claim 258, further comprising clearing the sample from the platform and introducing a carrier liquid to the first surface before the lysing step and after the step (b), wherein the analyte is released into the carrier liquid to form a lysate during the lysing step.

262. The method of claim 261, further comprising incubating the lysate with the sensor thereby associating the analyte with the analyte detecting agent and then clearing the lysate from the platform.

263. The method of claim 248, wherein the sensor comprises a plasmonic sensor.

264. The method of claim 248, wherein the sample is selected from the group consisting of a biological sample obtained from a subject, an environmental sample obtained from an environment, a swab sample, and combinations thereof.

265. The method of claim 248, wherein the analyte is selected from the group consisting of nucleotides, oligonucleotides, wild-type nucleotides, mutated nucleotides, double-stranded nucleotides, RNA, DNA, ribosomal RNA (rRNA), messenger RNA (mRNA), microDNA, microRNA, extrachromosomal circular DNA (eccDNA), cell free DNA (cfDNA), circulating tumor DNA (ctDNA), small molecules, proteins, mutated versions thereof, and combinations thereof.

266. The method of claim 248, wherein the analyte detecting agent is selected from the group consisting of aptamers, oligonucleotides, single-stranded oligonucleotides, double-stranded oligonucleotides, DNA, RNA, single stranded DNA, antibodies, peptide nucleic acids (PNAs), and combinations thereof.

267. The method of claim 248, wherein the change in property is characterized by a change in absorbance of the surface, a shift in peak absorbance wavelength of the surface, a change in plasmonic field intensity of the surface, enhanced resonance sensitivity, a color change in dark field image from the surface, a change in an image of the surface, a shortening of the analyte detecting agent, a change in measured light absorbance, a change in transmittance, a change in reflectance, a change in extinction, and combinations thereof.

268. The method of claim 248, wherein the second surface is in a form of an array, wherein the array comprises a plurality of different analyte detecting agents that are specific for different analytes, and wherein the method is utilized to detect a plurality of different analytes.
